(19) United States
(12) Patent Application Publication
(10) Pub. No.: US 2025/0306021 A1
(43) Pub. Date: Oct. 2, 2025

(54) Sero-Reactive Antigens for Coccidioidomycosis

(71) Applicants: Mitch Magee, Chandler, AZ (US); Douglas Lake, Scottsdale, AZ (US)

(72) Inventors: Mitch Magee, Chandler, AZ (US); Douglas Lake, Scottsdale, AZ (US)

(73) Assignee: Arizona Board of Regents on behalf of Arizona State University, Scottsdale, AZ (US)

(21) Appl. No.: 19/095,876

(22) Filed: Mar. 31, 2025

Related U.S. Application Data
(60) Provisional application No. 63/571,524, filed on Mar. 29, 2024.

(51) Int. Cl. G01N 33/569; G01N 33/543

(52) U.S. Cl. CPC G01N 33/56961; G01N 33/54386; G01N 2333/37; G01N 2469/10

(57) ABSTRACT

The present invention provides devices and methods for diagnosing and treating Coccidioides infection (valley fever). In some embodiments, the invention provides methods for identifying antigens useful in preparing similar devices for diagnosis of other pathogens of interest.

### CROSS REFERENCE TO RELATED APPLICATIONS

This application claims priority to U.S. Provisional Application No. 63/571,524, filed Mar. 29, 2024, which is hereby incorporated by reference herein in its entirety.

### STATEMENT REGARDING FEDERALLY SPONSORED RESEARCH

This invention was made with government support under R01AI155954 awarded by the National Institutes of Health. The government has certain rights in the invention.

### BACKGROUND OF THE INVENTION

Coccidioidomycosis is a fungal infection caused by Coccidioides, which is found in soil across much of the southwestern United States and northern Mexico. Disruption of soil, by wind, farming, and construction introduces Coccidioides spores into the air, where it can be inhaled and cause disease in humans and animals alike. Although often mild (valley fever), coccidioidomycosis can develop into more sever pathologies, including meningitis. Current methods for diagnosing valley fever include enzyme immunoassay (ELISA), immunodiffusion, and complement fixation tests, each of which rely on Coccidioides lysates that vary in sero-reactivity, producing lot-to-lot variation in test preparation, diagnosis, and patient prognosis. Accordingly, there is a need for a uniform, quantifiable method of producing consistently sero-reactive tests for infection with Coccidioides. The present invention addresses this long felt, but unmet, need.

### SUMMARY OF THE INVENTION

In some embodiments, the present invention provides methods of testing a subject for valley fever comprising the steps of: a) obtaining a serum sample from the subject; b) contacting the serum sample with one or more proteins selected from the group consisting of: woronin body major protein (A0A0E1S226), protein phosphatase 2C (J3K9P7), endochitinase-1/complement fixation (CF/CTS1/Endochitinase-1) (Q1E3R8), NADPH-cytochrome P450 reductase (J3KGJ4), proteasome component PUP2 (J3K500), ATP synthetase subunit 2, mitochondrial (J3KOX4), peroxisomal matrix protein (J3K6V5), elongation factor 1 gamma domain-containing protein (J3KLX5), 4-hydroxyphenylpyruvate dioxygenase (Q1E803), endo-1,3-beta-glucanase (J3KEN5), hsp90-like protein (J3KE37), ubiquitin-40S ribosomal protein S31 fusion protein (J3KC68), polyubiquitin (A0A0D8JXR9/A0A0D8JVA2), methylcrotonoyl-CoA carboxylase subunit beta (J3K8SO), calnexin (J3KHD0), GTP-binding protein YchF (A0A0E1RUI9), polyubiquitin (J3KHA0), 14-3-3-like protein (J3KA35), and hypothetical protein (titin/Nucleoporin homologue) (E9D7J3); c) detecting an interaction between an antibody in the serum and the one or more proteins; thereby detecting the presence of the antibody in the serum of the subject; and d) determining that the subject has valley fever when an interaction is detected.

In some embodiments, the one or more proteins are selected from the group consisting of: endochitinase-1/complement fixation (CF/CTS1/Endochitinase-1) (Q1E3R8), endo-1,3-beta-glucanase (J3KEN5), peroxisomal matrix protein (J3K6V5), hypothetical protein (titin/Nucleoporin homologue) (E9D7J3), hsp90-like protein (J3KE37), protein phosphatase 2C (J3K9P7), and polyubiquitin (A0A0D8JXR9/A0A0D8JVA2).

In some embodiments, the one or more proteins are selected from a group consisting of: endochitinase-1/complement fixation (CF/CTS1/Endochitinase-1) (Q1E3R8), endo-1,3-beta-glucanase (J3KEN5), peroxisomal matrix protein (J3K6V5), and hypothetical protein (titin/Nucleoporin homologue) (E9D7J3).

In some embodiments, in step b) the serum sample is contacted with a plurality of the one or more proteins.

In some embodiments, it is determined that the subject has valley fever when at least one of the one or more proteins interact with an antibody in the serum sample.

In some embodiments, it is determined that the subject has valley fever when an increase in the amount of the antibody is detected, relative to a reference level.

In some embodiments, the interaction between the serum and the one or more proteins is detected using a lateral flow immunoassay, an Enzyme-Linked Immunosorbent Assay (ELISA), an immunodiffusion assay, an immunosensor assay, a Nucleic Acid Programmable Protein Array (NAPPA), and an immunofluorescence assay.

In some embodiments, the method further comprises step e) administering a therapeutically effective amount of an antifungal composition to the subject.

In some embodiments, the antifungal composition comprises one or more selected from the group consisting of fluconazole, itraconazole, amphotericin B, voriconazole, posaconazole, isavuconazole, ketoconazole, terbinafine, nikkomycin Z, olorofim, and pharmaceutically acceptable salts and hydrates thereof.

In some embodiments, the present invention provides a diagnostic testing device for detecting Valley fever comprising a solid substrate decorated with a plurality of proteins, wherein the proteins are one or more proteins associated with Coccidioides posadasii infection.

In some embodiments, the one or more proteins are selected from the group consisting of: woronin body major protein (A0A0E1S226), protein phosphatase 2C (J3K9P7), endochitinase-1/complement fixation (CF/CTS1/Endochitinase-1) (Q1E3R8), NADPH-cytochrome P450 reductase (J3KGJ4), proteasome component PUP2 (J3K500), ATP synthetase subunit 2, mitochondrial (J3KOX4), peroxisomal matrix protein (J3K6V5), elongation factor 1 gamma domain-containing protein (J3KLX5), 4-hydroxyphenylpyruvate dioxygenase (Q1E803), endo-1,3-beta-glucanase (J3KEN5), hsp90-like protein (J3KE37), ubiquitin-40S ribosomal protein S31 fusion protein (J3KC68), polyubiquitin (A0A0D8JXR9/A0A0D8JVA2), methylcrotonoyl-CoA carboxylase subunit beta (J3K8SO), calnexin (J3KHD0), GTP-binding protein YchF (A0A0E1RUI9), polyubiquitin (J3KHA0), 14-3-3-like protein (J3KA35), and hypothetical protein (titin/Nucleoporin homologue) (E9D7J3).

In some embodiments, the one or more proteins are selected from the group consisting of: endochitinase-1/complement fixation (CF/CTS1/Endochitinase-1) (Q1E3R8), endo-1,3-beta-glucanase (J3KEN5), peroxisomal matrix protein (J3K6V5), hypothetical protein (titin/Nucleoporin homologue) (E9D7J3), hsp90-like protein (J3KE37), protein phosphatase 2C (J3K9P7), and polyubiquitin (A0A0D8JXR9/A0A0D8JVA2).

In some embodiments, the one or more proteins are selected from a group consisting of: endochitinase-1/complement fixation (CF/CTS1/Endochitinase-1) (Q1E3R8), endo-1,3-beta-glucanase (J3KEN5), peroxisomal matrix protein (J3K6V5), and hypothetical protein (titin/Nucleoporin homologue) (E9D7J3).

In some embodiments, the solid substrate is decorated with a plurality different proteins.

In some embodiments, the device is used in an assay selected from the group consisting of: a lateral flow immunoassay, an Enzyme-Linked Immunosorbent Assay (ELISA), an immunodiffusion assay, an immunosensor assay, a Nucleic Acid Programmable Protein Array (NAPPA), and an immunofluorescence assay.

In some embodiments, the present invention provides methods of identifying antigens associated with a pathogen comprising the steps of: a) obtaining a number of serum samples from subjects infected with the pathogen of interest and a number of serum samples from uninfected control subjects; b) contacting the serum samples with a plurality of proteins expressed by the pathogen of interest; c) detecting interactions between the proteins expressed by the pathogen of interest and antibodies present in the serum samples; and d) determining that a protein expressed by the pathogen of interest is an antigen when the protein interacts with antibodies present in the infected samples but not the uninfected samples.

In some embodiments, the plurality of proteins are immobilized on a solid substrate.

In some embodiments, in step c) interactions between the proteins and antibodies are detected by contacting the proteins and antibodies with fluorescently labeled secondary antibodies.

In some embodiments, the solid substrate comprises a plurality of different proteins expressed by the pathogen of interest.

In some embodiments, the present invention provides methods of testing a subject for valley fever comprising the steps of: a) obtaining a serum sample from the subject; b) contacting the serum sample with one or more antibodies that target or bind to one or more of the proteins selected from the group consisting of: woronin body major protein (A0A0E1S226), protein phosphatase 2C (J3K9P7), endochitinase-1/complement fixation (CF/CTS1/Endochitinase-1) (Q1E3R8), NADPH-cytochrome P450 reductase (J3KGJ4), proteasome component PUP2 (J3K500), ATP synthetase subunit 2, mitochondrial (J3KOX4), peroxisomal matrix protein (J3K6V5), elongation factor 1 gamma domain-containing protein (J3KLX5), 4-hydroxyphenylpyruvate dioxygenase (Q1E803), endo-1,3-beta-glucanase (J3KEN5), hsp90-like protein (J3KE37), ubiquitin-40S ribosomal protein S31 fusion protein (J3KC68), polyubiquitin (A0A0D8JXR9/A0A0D8JVA2), methylcrotonoyl-CoA carboxylase subunit beta (J3K8SO), calnexin (J3KHD0), GTP-binding protein YchF (A0A0E1RUI9), polyubiquitin (J3KHA0), 14-3-3-like protein (J3KA35), and hypothetical protein (titin/Nucleoporin homologue) (E9D7J3); c) detecting an interaction between a protein in the serum and the one or more antibodies; thereby detecting the presence of the protein in the serum of the subject; and d) determining that the subject has valley fever when an interaction is detected.

In some embodiments, the one or more antibodies target or bind to one or more of the proteins selected from the group consisting of: endochitinase-1/complement fixation (CF/CTS1/Endochitinase-1) (Q1E3R8), endo-1,3-beta-glucanase (J3KEN5), peroxisomal matrix protein (J3K6V5), hypothetical protein (titin/Nucleoporin homologue) (E9D7J3), hsp90-like protein (J3KE37), protein phosphatase 2C (J3K9P7), and polyubiquitin (A0A0D8JXR9/A0A0D8JVA2).

In some embodiments, the one or more antibodies target or bind to one or more of the proteins selected from a group consisting of: endochitinase-1/complement fixation (CF/CTS1/Endochitinase-1) (Q1E3R8), endo-1,3-beta-glucanase (J3KEN5), peroxisomal matrix protein (J3K6V5), and hypothetical protein (titin/Nucleoporin homologue) (E9D7J3).

In some embodiments, in step b) the serum sample is contacted with a plurality of the one or more antibodies.

In some embodiments, it is determined that the subject has valley fever when at least one of the one or more antibodies interact with a protein in the serum sample.

In some embodiments, it is determined that the subject has valley fever when an increase in the amount of the protein is detected, relative to a reference level.

In some embodiments, the interaction between the serum and the one or more antibodies is detected using a lateral flow immunoassay, an Enzyme-Linked Immunosorbent Assay (ELISA), an immunodiffusion assay, an immunosensor assay, a Nucleic Acid Programmable Protein Array (NAPPA), and an immunofluorescence assay.

In some embodiments, the method further comprises step e) administering a therapeutically effective amount of an antifungal composition to the subject.

In some embodiments, the antifungal composition comprises one or more selected from the group consisting of fluconazole, itraconazole, amphotericin B, voriconazole, posaconazole, isavuconazole, ketoconazole, terbinafine, nikkomycin Z, olorofim, and pharmaceutically acceptable salts and hydrates thereof.

In some embodiments, the present invention provides a diagnostic testing device for detecting Valley fever comprising a solid substrate decorated with a plurality of antibodies, wherein the one or more antibodies target or bind to one or more proteins associated with Coccidioides posadasii infection.

In some embodiments, the one or more antibodies target or bind to one or more of the proteins selected from the group consisting of: woronin body major protein (A0A0E1S226), protein phosphatase 2C (J3K9P7), endochitinase-1/complement fixation (CF/CTS1/Endochitinase-1) (Q1E3R8), NADPH-cytochrome P450 reductase (J3KGJ4), proteasome component PUP2 (J3K500), ATP synthetase subunit 2, mitochondrial (J3KOX4), peroxisomal matrix protein (J3K6V5), elongation factor 1 gamma domain-containing protein (J3KLX5), 4-hydroxyphenylpyruvate dioxygenase (Q1E803), endo-1,3-beta-glucanase (J3KEN5), hsp90-like protein (J3KE37), ubiquitin-40S ribosomal protein S31 fusion protein (J3KC68), polyubiquitin (A0A0D8JXR9/A0A0D8JVA2), methylcrotonoyl-CoA carboxylase subunit beta (J3K8SO), calnexin (J3KHD0), GTP-binding protein YchF (A0A0E1RUI9), polyubiquitin (J3KHA0), 14-3-3-like protein (J3KA35), and hypothetical protein (titin/Nucleoporin homologue) (E9D7J3).

In some embodiments, the one or more antibodies target or bind to one or more of the proteins selected from the group consisting of: endochitinase-1/complement fixation (CF/CTS1/Endochitinase-1) (Q1E3R8), endo-1,3-beta-glucanase (J3KEN5), peroxisomal matrix protein (J3K6V5), hypothetical protein (titin/Nucleoporin homologue) (E9D7J3), hsp90-like protein (J3KE37), protein phosphatase 2C (J3K9P7), and polyubiquitin (A0A0D8JXR9/A0A0D8JVA2).

In some embodiments, the one or more antibodies target or bind to one or more of the proteins selected from a group consisting of: endochitinase-1/complement fixation (CF/CTS1/Endochitinase-1) (Q1E3R8), endo-1,3-beta-glucanase (J3KEN5), peroxisomal matrix protein (J3K6V5), and hypothetical protein (titin/Nucleoporin homologue) (E9D7J3).

In some embodiments, the solid substrate is decorated with a plurality different antibodies.

In some embodiments, the device is used in an assay selected from the group consisting of: a lateral flow immunoassay, an Enzyme-Linked Immunosorbent Assay (ELISA), an immunodiffusion assay, an immunosensor assay, a Nucleic Acid Programmable Protein Array (NAPPA), and an immunofluorescence assay.

In some embodiments, the present invention provides methods of identifying antigens associated with a pathogen comprising the steps of: a) obtaining a number of serum samples from subjects infected with the pathogen of interest and a number of serum samples from uninfected control subjects; b) contacting the serum samples with a plurality of antibodies that target or bind to one or more proteins associated with the pathogen of interest; c) detecting interactions between the antibodies and the one or more proteins associated with the pathogen of interest present in the serum samples; and d) determining that a protein associated with the pathogen of interest is an antigen when the antibodies interact with proteins present in the infected samples but not the uninfected samples.

In some embodiments, the plurality of antibodies are immobilized on a solid substrate.

In some embodiments, in step c) interactions between the antibodies and proteins are detected by contacting the antibodies and proteins with fluorescently labeled secondary antibodies.

In some embodiments, the solid substrate comprises a plurality of different antibodies that target or bind to one or more proteins that are associated with the pathogen of interest.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 depicts a schematic representation of the various plasmids that can be obtained from the master plasmid utilized.

FIG. 2 depicts a schematic representation of a Nucleic Acid-Programmable Protein Array (NAPPA). Capture antibodies (e.g., anti-glutathione S-transferase (GST) antibodies) and plasmids encoding the protein of interest with a GST tag are printed into the nanowells (top). The proteins of interest are synthesized in situ and captured by the capture antibody upon addition of a medium comprising the requisite transcriptional and translational machinery, such as a HeLa cell lysate, and incubation (middle). After removal of the transcriptional/translational medium, the sample is incubated in the nanowell, allowing, for example, a serum antibody to bind to the protein expressed in the nanowell. Secondary fluorescent antibodies are then added, such as fluorescently tagged anti-canine IgG antibodies, non-binding antibodies are washed away, and the plate is read (bottom).

FIG. 3 depicts representative images of NAPPA plates reacted with complementary fixation (CF)-positive serum (top) and CF-negative serum (bottom) with the IgG control displayed in the respective boxes. Upon scanning, signal intensity is normalized to the median intensity.

FIG. 4 depicts representative normalized fluorescence output for NAPPA assay results of CF-positive and CF-negative serum with coccidioidin lysate (CDN-L) and coccidioidin filtrate (CND-F) extracts.

FIG. 5 depicts representative normalized fluorescence output for NAPPA assay results of CF-positive and CF-negative serum reacted with complement fixation (CF) antigen (CTS-1).

FIG. 6 depicts representative normalized fluorescence output for NAPPA assay results of serum from laboratory-acquired infected animals reacting with CTS-1 over time. Reactivity to CTS-1 is not observed until at least 4-weeks post-infection.

FIG. 7 depicts a representative image of NAPPA plate reacted with CF-positive serum.

FIG. 8 depicts a representative Venn diagram of antigens reactive with serum obtained from canines with community-acquired or laboratory-acquired infections. Sixteen antigens were observed exclusively in community-acquired samples while 35 antigens were exclusively observed to react with laboratory-acquired infection samples, with 20 antigens commonly shared.

FIG. 9A and FIG. 9B depicts a representative diagnostic model based on one of the 20 commonly observed antigens. FIG. 9A depicts a representative ROC curve for a diagnostic model predicting Coccidioides infection based on serum reactivity with hypothetical protein (titin/Nucleoporin homologue) CIMG_07502. FIG. 9B depicts a representative plot of reactivity of serum from animals at the day of infection (TO) lacking a response to the hypothetical protein and the reactivity of serum from animals diagnosed as infected based on the model 2-weeks post-infection (T2).

FIG. 10 depicts representative data demonstrating CF/CTS1 IgG Reactivity. Human samples were identified by one or more serological assays to obtain laboratory negative (LN) or laboratory positive (LP) samples. The first column on the left demonstrates laboratory negative samples, the green circles denote laboratory negative samples that were detected positive by NAPPA for CF/CTS1 (representing False Negative Diagnosis). The second column on the graph on the left are laboratory positive samples that are primarily positive to CF/CTS1 by NAPPA. The circles in red are patient samples that were classified as positive but were negative in reactivity to CF/CTS1. Thus, they are likely reactive to other protein components in the complex fungal antigen preparations. In the graph on the right, the laboratory positives were broken down by type of immunoassay performed. The CF Titer assay was the traditional complement fixation test. The second group was positive by both EIA and ID. The third group was positive by EIA only. Any sample represented by a red circle was negative to CF/CTS1 by NAPPA.

FIG. 11 depicts representative data demonstrating J3K6V5 IgG Reactivity using a variety of immunoassays.

FIG. 12 depicts representative data demonstrating J3KE37 (left) and J3K9P7 (right) IgG Reactivity using a variety of immunoassays.

FIG. 13 depicts representative data demonstrating J3KEN5 (left) and A0A0D8JXR9/A0A0D8JVA2 (right) IgG Reactivity using a variety of immunoassays.

### DETAILED DESCRIPTION

The present invention is based, in part, on the unexpected discovery that several dozen proteins expressed by Coccidioides posadasii are sero-reactive against all tested samples of sera from subjects infected with Coccidioides. Thus, in one aspect, the present invention provides a diagnostic testing device or assay comprising these antigens for diagnosing valley fever. In some embodiments, the invention provides methods of diagnosing a subject with valley fever through the use of a diagnostic testing device of the present invention. In some embodiments, the invention provides methods of determining the prognosis of a patient that has valley fever. Consequently, in some embodiments, the invention provides methods of treating valley fever in subjects diagnosed accordingly.

In another aspect, the present invention relates to a method for identifying antigens of a pathogen of interest that are useful for designing diagnostics for that pathogen.

### Definitions

Unless defined otherwise, all technical and scientific terms used herein have the same meaning as commonly understood by one of ordinary skill in the art to which this invention belongs. Any methods and materials similar or equivalent to those described herein can be used in the practice or testing of the present invention.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value, for example numerical values and/or ranges, such as an amount, a temporal duration, and the like, is meant to encompass variations of ±20%, ±10%, ±5%, ±1%, or ±0.1% from the specified value, as such variations are appropriate to perform the disclosed methods. For example, “about 40 [units]” may mean within ±25% of 40 (e.g., from 30 to 50), within ±20%, ±15%, ±10%, ±9%, ±8%, ±7%, ±6%, ±5%, ±4%, ±3%, ±2%, ±1%, less than ±1%, or any other value or range of values therein or therebelow. Furthermore, the phrases “less than about [a value]” or “greater than about [a value]” should be understood in view of the definition of the term “about” provided herein.

The term “label” when used herein refers to a detectable compound or composition that is conjugated directly or indirectly to a probe to generate a “labeled” probe. The label may be detectable by itself (e.g., radioisotope labels or fluorescent labels) or, in the case of an enzymatic label, may catalyze chemical alteration of a substrate compound or composition that is detectable (e.g., avidin-biotin). In some instances, primers can be labeled to detect a PCR product.

As used herein, an “immunoassay” refers to any binding assay that uses an antibody capable of binding specifically to a target molecule to detect and quantify the target molecule.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more other species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

To “treat” a disease as the term is used herein, means to reduce the frequency or severity of at least one sign or symptom of a disease or disorder experienced by a subject.

As used herein, “treating a disease or disorder” means reducing the frequency with which a symptom of the disease or disorder is experienced by a patient. Disease and disorder are used interchangeably herein.

A disease or disorder is “alleviated” if the severity of a sign or symptom of the disease or disorder, the frequency with which such a sign or symptom is experienced by a patient, or both, is reduced.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject.

As used herein, the term “diagnosis” refers to the determination of the presence of a disease or disorder. In some embodiments of the present invention, methods for making a diagnosis are provided which permit determination of the presence of a particular disease or disorder.

The term “abnormal” when used in the context of organisms, tissues, cells, or components thereof, refers to those organisms, tissues, cells, or components thereof that differ in at least one observable or detectable characteristic (e.g., age, treatment, time of day, etc.) from those organisms, tissues, cells, or components thereof that display the “normal” (expected) respective characteristic. Characteristics which are normal or expected for one cell or tissue type, might be abnormal for a different cell or tissue type.

The term “antibody,” as used herein, refers to an immunoglobulin molecule, which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)₂, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. κ and λ light chains refer to the two major antibody light chain isotypes.

By the term “synthetic antibody” as used herein, is meant an antibody, which is generated using recombinant DNA technology, such as, for example, an antibody expressed by a bacteriophage. The term should also be construed to mean an antibody, which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology, which is available and well known in the art. The term should also be construed to mean an antibody, which has been generated by the synthesis of an RNA molecule encoding the antibody. The RNA molecule expresses an antibody protein, or an amino acid sequence specifying the antibody, wherein the RNA has been obtained by transcribing DNA (synthetic or cloned) or other technology, which is available and well known in the art.

The term “antigen” or “Ag” as used herein is defined as a molecule that provokes an adaptive immune response. This immune response may involve either antibody production, or the activation of specific immunogenically-competent cells, or both. The skilled artisan will understand that any macromolecule, including virtually all proteins or peptides, can serve as an antigen. Furthermore, antigens can be derived from recombinant or genomic DNA or RNA. A skilled artisan will understand that any DNA or RNA, which comprises a nucleotide sequence or a partial nucleotide sequence encoding a protein that elicits an adaptive immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared X 100. For example, if 6 of 10 of the positions in two sequences are matched or homologous then the two sequences are 60% homologous. Generally, a comparison is made when two sequences are aligned to give maximum homology.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

In certain instances, the polynucleotide or nucleic acid of the invention is a “nucleoside-modified nucleic acid,” which refers to a nucleic acid comprising at least one modified nucleoside. A “modified nucleoside” refers to a nucleoside with a modification. For example, over one hundred different nucleoside modifications have been identified in RNA (Rozenski, et al., 1999, The RNA Modification Database: 1999 update. Nucl Acids Res 27:196-197).

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

As used herein, the terms “amino acid”, “amino acidic monomer”, or “amino acid residue” refer to any of the twenty naturally occurring amino acids including synthetic amino acids with unnatural side chains and including both D and L optical isomers.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

A “fragment” of a peptide sequence or a nucleic acid sequence that encodes an antigen may be 100% identical to the full length except missing at least one amino acid or at least one nucleotide from the 5′ and/or 3′ end, in each case with or without sequences encoding signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length coding sequence, excluding any heterologous signal peptide added. The fragment may comprise a fragment that encode a polypeptide that is 95% or more, 96% or more, 97% or more, 98% or more, or 99% or more identical to the antigen and additionally optionally comprise sequence encoding an N terminal methionine or heterologous signal peptide, which is not included when calculating percent identity.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Diagnostic Testing Device

In one aspect, the present invention provides solid substrate-based diagnostic devices and methods of use thereof. The devices are intended to provide a reproducible means of testing a subject's serum for antibodies against Coccidioides proteins, thereby identifying subjects that are, or have been, infected.

TABLE 1
Identified antigens

| | UniProt | | GenBank RefSeq |
|---|---|---|---|
| Gene Symbol | Accession No. | Gene Name | Accession |
| CIMG_06738 | A0A0E1S226 | woronin body major protein | XP_001242842.2 |
| CIMG_07139 | J3K9P7 | protein phosphatase 2C | XP_001243243.2 |
| CIMG_02795 | Q1E3R8 | endochitinase-1/complement fixation* | XP_001249024.1 |
| CIMG_00220 | J3KGJ4 | NADPH-cytochrome P450 reductase | XP_001246449.1 |
| CIMG_08182 | J3K500 | proteasome component PUP2 | XP_001241019.1 |
| CIMG_10149 | J3KOX4 | ATP synthetase subunit 2, mitochondrial | XP_001239127.1 |
| CIMG_05828 | J3K6V5 | peroxisomal matrix protein* | XP_001241932.1 |
| CIMG_02699 | J3KLX5 | elongation factor 1 gamma domain- | XP_001248928.1 |
| | | containing protein | |
| CIMG_01310 | Q1E803 | 4-hydroxyphenylpyruvate dioxygenase* | XP_001247539.1 |
| CIMG_04988 | J3KEN5 | endo-1,3-beta-glucanase | XP_001245547.2 |
| CIMG_04729 | J3KE37 | hsp90-like protein | XP_001245288.1 |
| CIMG_03821 | J3KC68 | ubiquitin-40S ribosomal protein S31 | XP_001244380.1 |
| | | fusion protein | |
| CIMG_11455 | A0A0D8JXR9; | polyubiquitin | XP_004446138.1 |
| | A0A0D8JVA2 | | |
| CIMG_06723 | J3K8S0 | methylcrotonoyl-CoA carboxylase | XP_00124827.1 |
| | | subunit beta | |
| CIMG_00613 | J3KHDO | calnexin | XP_001246842.1 |
| CIMG_10273 | AOAOE1RUI9 | GTP-binding protein YchF | XP_001239251.1 |
| CIMG_12861 | J3KHA0 | polyubiquitin | XP_001246797.1 |
| CIMG_07320 | J3KA35 | 14-3-3-like protein | XP_001243424.1 |
| CPSG_05795 | E9D7J3 | hypothetical protein (titin/Nucleoporin | EFW17352.1 |
| | | homologue) | |

*Known immunoreactive protein

In one embodiment, the present invention comprises a device comprising a solid substrate which is decorated with one or more Coccidioides-associated proteins. For example, in certain embodiments, the device comprises one or more Coccidioides-associated proteins immobilized or bound to the solid substrate.

In some embodiments, the Coccidioides-associated proteins are proteins expressed by Coccidioides that have been identified as sero-reactive antigens. In some embodiments, the one or more Coccidioides-associated proteins comprise one or more of the proteins of Table 1. In some embodiments, the proteins are one or more selected from the group consisting of: woronin body major protein (A0A0E1S226), protein phosphatase 2C (J3K9P7), endochitinase-1/complement fixation (CF/CTS1/Endochitinase-1) (Q1E3R8), NADPH-cytochrome P450 reductase (J3KGJ4), proteasome component PUP2 (J3K500), ATP synthetase subunit 2, mitochondrial (J3KOX4), peroxisomal matrix protein (J3K6V5), elongation factor 1 gamma domain-containing protein (J3KLX5), 4-hydroxyphenylpyruvate dioxygenase (Q1E803), endo-1,3-beta-glucanase (J3KEN5), hsp90-like protein (J3KE37), ubiquitin-40S ribosomal protein S31 fusion protein (J3KC68), polyubiquitin (A0A0D8JXR9/A0A0D8JVA2), methylcrotonoyl-CoA carboxylase subunit beta (J3K8SO), calnexin (J3KHD0), GTP-binding protein YchF (A0A0E1RUI9), polyubiquitin (J3KHA0), 14-3-3-like protein (J3KA35), and hypothetical protein (titin/Nucleoporin homologue) (E9D7J3). In some embodiments, the proteins are one or more selected from the group consisting of: endochitinase-1/complement fixation (CF/CTS1/Endochitinase-1) (Q1E3R8), endo-1,3-beta-glucanase (J3KEN5), peroxisomal matrix protein (J3K6V5), hypothetical protein (titin/Nucleoporin homologue) (E9D7J3), hsp90-like protein (J3KE37), protein phosphatase 2C (J3K9P7), and polyubiquitin (A0A0D8JXR9/A0A0D8JVA2). In some embodiments, the proteins are one or more selected from the group consisting of: endochitinase-1/complement fixation (CF/CTS1/Endochitinase-1) (Q1E3R8), endo-1,3-beta-glucanase (J3KEN5), peroxisomal matrix protein (J3K6V5), and hypothetical protein (titin/Nucleoporin homologue) (E9D7J3).

In some embodiments, the device is decorated with 1, 2, 3, 4, 5, 6, 7, 8, 9, 10, 11, 12, 13, 14, 15, 16, 17, 18, 19, or 20 of the proteins selected from the group consisting of: woronin body major protein (A0A0E1S226), protein phosphatase 2C (J3K9P7), endochitinase-1/complement fixation (CF/CTS1/Endochitinase-1) (Q1E3R8), NADPH-cytochrome P450 reductase (J3KGJ4), proteasome component PUP2 (J3K500), ATP synthetase subunit 2, mitochondrial (J3KOX4), peroxisomal matrix protein (J3K6V5), elongation factor 1 gamma domain-containing protein (J3KLX5), 4-hydroxyphenylpyruvate dioxygenase (Q1E803), endo-1,3-beta-glucanase (J3KEN5), hsp90-like protein (J3KE37), ubiquitin-40S ribosomal protein S31 fusion protein (J3KC68), polyubiquitin (A0A0D8JXR9/A0A0D8JVA2), methylcrotonoyl-CoA carboxylase subunit beta (J3K8SO), calnexin (J3KHD0), GTP-binding protein YchF (A0A0E1RUI9), polyubiquitin (J3KHA0), 14-3-3-like protein (J3KA35), and hypothetical protein (titin/Nucleoporin homologue) (E9D7J3). In some embodiments, the device is decorated with 1, 2, 3, 4, 5, 6, or 7, of the proteins selected from the group consisting of: endochitinase-1/complement fixation (CF/CTS1/Endochitinase-1) (Q1E3R8), endo-1,3-beta-glucanase (J3KEN5), peroxisomal matrix protein (J3K6V5), hypothetical protein (titin/Nucleoporin homologue) (E9D7J3), hsp90-like protein (J3KE37), protein phosphatase 2C (J3K9P7), and polyubiquitin (A0A0D8JXR9/A0A0D8JVA2). In some embodiments, the device is decorated with 1, 2, 3, or 4, of the proteins selected from the group consisting of: endochitinase-1/complement fixation (CF/CTS1/Endochitinase-1) (Q1E3R8), endo-1,3-beta-glucanase (J3KEN5), peroxisomal matrix protein (J3K6V5), and hypothetical protein (titin/Nucleoporin homologue) (E9D7J3).

In some embodiments, the device comprises seven or more proteins. In some embodiments, the seven or more proteins are selected from the group consisting of: endochitinase-1/complement fixation (CF/CTS1/Endochitinase-1) (Q1E3R8), endo-1,3-beta-glucanase (J3KEN5), peroxisomal matrix protein (J3K6V5), hypothetical protein (titin/Nucleoporin homologue) (E9D7J3), hsp90-like protein (J3KE37), protein phosphatase 2C (J3K9P7), and polyubiquitin (A0A0D8JXR9/A0A0D8JVA2). In some embodiments, the device comprises four or more proteins. In some embodiments, the four or more proteins are selected from the group consisting of: endochitinase-1/complement fixation (CF/CTS1/Endochitinase-1) (Q1E3R8), endo-1,3-beta-glucanase (J3KEN5), peroxisomal matrix protein (J3K6V5), and hypothetical protein (titin/Nucleoporin homologue) (E9D7J3). In some embodiments, the device comprises at least one protein, wherein the at least one protein is endochitinase-1/complement fixation (CF/CTS1/Endochitinase-1) (Q1E3R8). In some embodiments, the device comprises at least one protein, wherein the at least one protein is endo-1,3-beta-glucanase (J3KEN5). In some embodiments, the device comprises at least one protein, wherein the at least one protein is peroxisomal matrix protein (J3K6V5). In some embodiments, the device comprises at least one protein, wherein the at least one protein is hypothetical protein (titin/Nucleoporin homologue) (E9D7J3). In some embodiments, the device comprises a first protein endochitinase-1/complement fixation (CF/CTS1/Endochitinase-1) (Q1E3R8), a second protein endo-1,3-beta-glucanase (J3KEN5), a third protein peroxisomal matrix protein (J3K6V5), and a fourth protein hypothetical protein (titin/Nucleoporin homologue) (E9D7J3).

In some embodiments, the one or more Coccidioides-associated proteins are antibodies that target or bind to a protein expressed by Coccidioides. In some embodiments, the one or more Coccidioides-associated proteins are antibodies that target or bind to one or more of the proteins of Table 1. In some embodiments, the antibodies are antibodies which target or bind one or more selected from the group consisting of: woronin body major protein (A0A0E1S226), protein phosphatase 2C (J3K9P7), endochitinase-1/complement fixation (CF/CTS1/Endochitinase-1) (Q1E3R8), NADPH-cytochrome P450 reductase (J3KGJ4), proteasome component PUP2 (J3K500), ATP synthetase subunit 2, mitochondrial (J3KOX4), peroxisomal matrix protein (J3K6V5), elongation factor 1 gamma domain-containing protein (J3KLX5), 4-hydroxyphenylpyruvate dioxygenase (Q1E803), endo-1,3-beta-glucanase (J3KEN5), hsp90-like protein (J3KE37), ubiquitin-40S ribosomal protein S31 fusion protein (J3KC68), polyubiquitin (A0A0D8JXR9/A0A0D8JVA2), methylcrotonoyl-CoA carboxylase subunit beta (J3K8SO), calnexin (J3KHD0), GTP-binding protein YchF (A0A0E1RUI9), polyubiquitin (J3KHA0), 14-3-3-like protein (J3KA35), and hypothetical protein (titin/Nucleoporin homologue) (E9D7J3). In some embodiments, the antibodies are antibodies which target one or more selected from the group consisting of: endochitinase-1/complement fixation (CF/CTS1/Endochitinase-1) (Q1E3R8), endo-1,3-beta-glucanase (J3KEN5), peroxisomal matrix protein (J3K6V5), hypothetical protein (titin/Nucleoporin homologue) (E9D7J3), hsp90-like protein (J3KE37), protein phosphatase 2C (J3K9P7), and polyubiquitin (A0A0D8JXR9/A0A0D8JVA2). In some embodiments, the antibodies are antibodies which target one or more selected from the group consisting of: endochitinase-1/complement fixation (CF/CTS1/Endochitinase-1) (Q1E3R8), endo-1,3-beta-glucanase (J3KEN5), peroxisomal matrix protein (J3K6V5), and hypothetical protein (titin/Nucleoporin homologue) (E9D7J3).

In some embodiments, the antibodies are monoclonal antibodies. In some embodiments, the antibodies are polyclonal antibodies. In some embodiments, the antibodies are functional fragments of antibodies. In some embodiments, the device is decorated with antibodies against 1, 2, 3, 4, 5, 6, 7, 8, 9, 10, 11, 12, 13, 14, 15, 16, 17, 18, 19, or 20 of the proteins selected from the group consisting of: woronin body major protein (A0A0E1S226), protein phosphatase 2C (J3K9P7), endochitinase-1/complement fixation (CF/CTS1/Endochitinase-1) (Q1E3R8), NADPH-cytochrome P450 reductase (J3KGJ4), proteasome component PUP2 (J3K500), ATP synthetase subunit 2, mitochondrial (J3KOX4), peroxisomal matrix protein (J3K6V5), elongation factor 1 gamma domain-containing protein (J3KLX5), 4-hydroxyphenylpyruvate dioxygenase (Q1E803), endo-1,3-beta-glucanase (J3KEN5), hsp90-like protein (J3KE37), ubiquitin-40S ribosomal protein S31 fusion protein (J3KC68), polyubiquitin (A0A0D8JXR9/A0A0D8JVA2), methylcrotonoyl-CoA carboxylase subunit beta (J3K8SO), calnexin (J3KHD0), GTP-binding protein YchF (A0A0E1RUI9), polyubiquitin (J3KHA0), 14-3-3-like protein (J3KA35), and hypothetical protein (titin/Nucleoporin homologue) (E9D7J3). In some embodiments, the device is decorated with antibodies against 1, 2, 3, 4, 5, 6, or 7, of the proteins selected from the group consisting of: endochitinase-1/complement fixation (CF/CTS1/Endochitinase-1) (Q1E3R8), endo-1,3-beta-glucanase (J3KEN5), peroxisomal matrix protein (J3K6V5), hypothetical protein (titin/Nucleoporin homologue) (E9D7J3), hsp90-like protein (J3KE37), protein phosphatase 2C (J3K9P7), and polyubiquitin (A0A0D8JXR9/A0A0D8JVA2). In some embodiments, the device is decorated with antibodies against 1, 2, 3, or 4, of the proteins selected from the group consisting of: endochitinase-1/complement fixation (CF/CTS1/Endochitinase-1) (Q1E3R8), endo-1,3-beta-glucanase (J3KEN5), peroxisomal matrix protein (J3K6V5), and hypothetical protein (titin/Nucleoporin homologue) (E9D7J3).

In a variety of embodiments, the solid substrate comprises a plurality of nanowells, each of which is decorated with a single type of protein. In some embodiments, there is one nanowell per protein type. In some embodiments, there are 2, 3, 4, 5, 6, 7, 8, 9, or 10 nanowells per protein as technical replicates. In some embodiments, the proteins are synthesized in situ in each nanowell. In certain embodiments, the diagnostic testing device is a Nucleic Acid-Programmable Protein Array (NAPPA). In some embodiments, each nanowell of the NAPPA comprises a nucleic acid encoding one of the proteins. In some embodiments, the nucleic acid encoding the protein encodes a protein modified to comprise an affinity tag for immobilization within the nanowell. In some embodiments, the affinity tag is a glutathione S-transferase (GST).

In some embodiments, the diagnostic testing device comprises a capture molecule. In some embodiments, the capture molecule is selected from one or more of the group consisting of: a protein, a peptide, an antibody, an antigen, an enzyme, a substrate, and a nucleic acid. In some embodiments, the capture molecule is a protein. In some embodiments, the capture molecule is a protein associated with Coccidioides posadasii. In some embodiments, the capture molecule binds to an antibody. In some embodiments, the diagnostic testing device comprises one or more capture molecules. In some embodiments, the one or more capture molecules is a protein selected from the group consisting of woronin body major protein (A0A0E1S226), protein phosphatase 2C (J3K9P7), endochitinase-1/complement fixation (CF/CTS1/Endochitinase-1) (Q1E3R8), NADPH-cytochrome P450 reductase (J3KGJ4), proteasome component PUP2 (J3K500), ATP synthetase subunit 2, mitochondrial (J3KOX4), peroxisomal matrix protein (J3K6V5), elongation factor 1 gamma domain-containing protein (J3KLX5), 4-hydroxyphenylpyruvate dioxygenase (Q1E803), endo-1,3-beta-glucanase (J3KEN5), hsp90-like protein (J3KE37), ubiquitin-40S ribosomal protein S31 fusion protein (J3KC68), polyubiquitin (A0A0D8JXR9/A0A0D8JVA2), methylcrotonoyl-CoA carboxylase subunit beta (J3K8S0), calnexin (J3KHD0), GTP-binding protein YchF (A0A0E1RUI9), polyubiquitin (J3KHA0), 14-3-3-like protein (J3KA35), and hypothetical protein (titin/Nucleoporin homologue) (E9D7J3). In some embodiments, the one or more capture molecules are selected from the group consisting of: endochitinase-1/complement fixation (CF/CTS1/Endochitinase-1) (Q1E3R8), endo-1,3-beta-glucanase (J3KEN5), peroxisomal matrix protein (J3K6V5), hypothetical protein (titin/Nucleoporin homologue) (E9D7J3), hsp90-like protein (J3KE37), protein phosphatase 2C (J3K9P7), and polyubiquitin (A0A0D8JXR9/A0A0D8JVA2). In some embodiments, the one or more capture molecules are selected from the group consisting of: endochitinase-1/complement fixation (CF/CTS1/Endochitinase-1) (Q1E3R8), endo-1,3-beta-glucanase (J3KEN5), peroxisomal matrix protein (J3K6V5), and hypothetical protein (titin/Nucleoporin homologue) (E9D7J3).

In some embodiments, the one or more capture molecules is an antibody which target or bind one or more selected from the group consisting of: woronin body major protein (A0A0E1S226), protein phosphatase 2C (J3K9P7), endochitinase-1/complement fixation (CF/CTS1/Endochitinase-1) (Q1E3R8), NADPH-cytochrome P450 reductase (J3KGJ4), proteasome component PUP2 (J3K500), ATP synthetase subunit 2, mitochondrial (J3KOX4), peroxisomal matrix protein (J3K6V5), elongation factor 1 gamma domain-containing protein (J3KLX5), 4-hydroxyphenylpyruvate dioxygenase (Q1E803), endo-1,3-beta-glucanase (J3KEN5), hsp90-like protein (J3KE37), ubiquitin-40S ribosomal protein S31 fusion protein (J3KC68), polyubiquitin (A0A0D8JXR9/A0A0D8JVA2), methylcrotonoyl-CoA carboxylase subunit beta (J3K8SO), calnexin (J3KHD0), GTP-binding protein YchF (A0A0E1RUI9), polyubiquitin (J3KHA0), 14-3-3-like protein (J3KA35), and hypothetical protein (titin/Nucleoporin homologue) (E9D7J3). In some embodiments, the one or more capture molecules is an antibody which target or bind one or more selected from the group consisting of: endochitinase-1/complement fixation (CF/CTS1/Endochitinase-1) (Q1E3R8), endo-1,3-beta-glucanase (J3KEN5), peroxisomal matrix protein (J3K6V5), hypothetical protein (titin/Nucleoporin homologue) (E9D7J3), hsp90-like protein (J3KE37), protein phosphatase 2C (J3K9P7), and polyubiquitin (A0A0D8JXR9/A0A0D8JVA2). In some embodiments, the one or more capture molecules is an antibody which target or bind one or more selected from the group consisting of: endochitinase-1/complement fixation (CF/CTS1/Endochitinase-1) (Q1E3R8), endo-1,3-beta-glucanase (J3KEN5), peroxisomal matrix protein (J3K6V5), and hypothetical protein (titin/Nucleoporin homologue) (E9D7J3).

In some embodiments, the diagnostic testing device uses an immunoassay detection system. Exemplary immunoassays can include direct Enzyme-Linked Immunosorbent Assay (ELISA), indirect ELISA, sandwich ELISA, competitive ELISA, multiplex ELISA, lateral flow immunoassay, immunodiffusion assay, immunosensor assay, immunofluorescence assay, radioimmunoassay, ELISPOT technologies or mass spectrometry analysis methods. In some embodiments, the immunoassay detection system is selected from the group consisting of: a lateral flow immunoassay and an Enzyme-Linked Immunosorbent Assay (ELISA). In some embodiments, the diagnostic testing device comprises an antibody-based detection system. In some embodiments, the diagnostic testing device comprises a multiplexed immunoassay.

The following examples of immunoassays describe detecting antigens within a sample, however these immunoassays can also be used to detect antibodies within a sample. Preferably, this invention provides methods of detecting antibodies within sample using capturing proteins, wherein the capturing proteins are Coccidioides proteins. The direct ELISA uses the method of directly measuring the binding of the target to a capture agent. For example, microwell plates are coated with a sample containing the target (e.g., antibody binding to protein associated with Coccidioides posadasii, or protein associated with Coccidioides posadasii) and the binding of target to the associated capture agent is quantitated by a colorimetric, chemiluminescent, fluorescent or electrical end-point. Since a secondary antibody step is omitted, the direct ELISA is relatively quick, and avoids potential problems of cross-reactivity of the secondary antibody with components in the sample. Direct methods can lack the additional signal amplification that can be achieved with the use of a secondary antibody.

The indirect, two-step ELISA method uses a labelled secondary antibody for detection. First, a capture agent is incubated with the target. This is followed by incubation with a labelled secondary antibody that recognizes the capture agent-target complex.

The sandwich ELISA measures the amount of a target between two layers of capture agents. The target to be measured must contain at least two binding sites, capable of binding to the capture agent, since at least two capture agents act in the sandwich. For this reason, sandwich assays are restricted to the quantitation of multivalent targets such as proteins or polysaccharides.

Multiplex ELISA is a microtiter plate ELISA-based protein array assay that allows simultaneous detection of multiple targets at multiple array addresses within a single well. For examples, the protein array comprises one or more proteins associated with Coccidioides posadasii. Different types of multiplex ELISA have been developed and are in practice. One of the examples is to measure antibodies by coating or printing capture proteins (e.g., protein associated with Coccidioides posadasii) in an array format within a single well to allow for the construction of “sandwich” ELISA quantification assays. Generally, multiplex ELISA can also be achieved through a capture agent array, where different capture agents can be attached to a solid phase e.g. a glass plate to capture corresponding antibodies in a biological sample. The detection method can be direct or indirect, sandwich or competitive, labelling or non-labelling, depending upon antibody array technologies.

The Enzyme-Linked Immunosorbent Spot (ELISpot) assay employs the sandwich assay approach of the Enzyme-Linked ImmunoSorbent Assay (ELISA), with some variations. The capture agent is coated aseptically onto a polyvinylidene difluoride (PVDF)-backed microwell plate. The plate is blocked with serum proteins, cells of interest are plated out at varying densities, along with antigen or mitogen, and plates are incubated at 37° C. Targets in a serum sample are captured locally by the coated antibody on the high surface area PVDF membrane. The wells are washed to remove cells, debris, and media components. A second capture agent (biotinylated) reactive with a distinct epitope of the target is employed to detect the captured target. The detected target is then visualized using avidin-HRP, and a precipitating substrate (e.g. AEC). The colored end product (spot) represents an individual expressing the target antigen. The spots can be counted manually (e.g., with a dissecting microscope) or using an automated reader to capture the microwell images and to analyze spot number and size.

Lateral flow immunoassays, also called lateral flow tests, dipsticks or simply strip tests, are simple one- or two-step assays for the qualitative determination of targets directly in liquid samples. Specific lines, or zones, are “striped” onto, or applied to, a test membrane that contains capture agents designed to react with and bind to predefined targets of interest that may be present in a liquid test sample. When a liquid sample is applied to one end of the test membrane, the sample is drawn by capillary action along the longitudinal axis of the membrane strip. Targets of interest present in the sample interact with the capture agents, producing measurable and detectable changes along the striped agent assay test zones. The benefits of lateral flow tests include: (a) they have a user-friendly format; (b) a very short time is required to obtain the test result; (c) they have long-term stability over a wide range of climates; and (d) they are relatively inexpensive to make. These features make strip tests ideal for applications such as home testing, rapid point-of-care testing, and testing in the field for various environmental and agricultural analytes. In addition, they provide reliable testing that might not otherwise be available in developing countries.

A rapid lateral flow test generally comprises of a system of overlapping porous materials containing the dried components needed to perform the test. These membranes are assembled in small strips, which can be placed into a plastic housing for ease in handling. Lateral flow tests can be used to detect any target that can be bound to a visually detectable capture agent attached to a solid support, both qualitatively and, in many cases, semi-quantitatively. A multiplexed lateral flow assay system can be used to detect multiple targets. A multiplexed lateral flow assay system aligns multiple lateral flow assays, or test strips, into a single large cassette. A liquid test sample is applied at a specific location and then divided and directed into multiple separate channels, with each channel containing capturing agents for detecting a specific target.

### Methods of Identifying Antigens

In certain aspects, the present invention provides methods of identifying antigens associated with a pathogen. In some embodiments, the method comprises the steps of:
a) obtaining a number of serum samples from subjects infected with the pathogen of interest and a number of serum samples from uninfected control subjects;
b) contacting the serum samples with a plurality of proteins expressed by the pathogen of interest;
c) detecting interactions between the proteins expressed by the pathogen of interest and antibodies present in the serum samples; and
d) determining that a protein expressed by the pathogen of interest is an antigen when the protein interacts with antibodies present in the infected samples but not the uninfected samples.

In one embodiment, the pathogen of interest is Coccidioides posadasii.

In some embodiments, the proteins expressed by the pathogen of interest are immobilized. In some embodiments, the proteins are immobilized on beads. In some embodiments, the proteins are immobilized on a planar solid substrate. In some embodiments, the proteins are immobilized within nanowells in a planar solid substrate. In some embodiments, step b) further comprises washing the immobilized proteins to remove unbound antibodies from the serum.

In some embodiments, the interactions of step c) are detected by optical means. In some embodiments, interaction of the proteins with one or more antibodies induces a conformational change in the antigen, resulting in an optically observable phenomenon (e.g., fluorescence). In some embodiments, the conformational change results in reduction or cessation of the observable phenomenon (e.g., fluorescence is no longer observed).

In some embodiments, interaction of the antigen with one or more antibodies enables binding of a second antibody. In some embodiments, the secondary antibody is fluorescently labeled. In some embodiments, the second antibody possesses an enzymatic function, wherein the activity of the enzyme results in an optically observable phenomenon (e.g., change in color). In some embodiments, step c) further comprises the step of washing the proteins to remove unbound secondary antibodies.

In some embodiments, the proteins expressed by the pathogen of interest are synthesized in situ. In some embodiments, the proteins are synthesized in a nanowell in a planar solid substrate. In some embodiments, the planar solid substrate is a Nucleic Acid Programmable Protein Array (NAPPA).

### Methods of Diagnosis and Treatment

In one aspect, the present invention provides methods of diagnosing, determining the prognosis of, and treating a subject with a disease or disorder. In some embodiments, the disease or disorder is associated with a pathogen. In some embodiments, the pathogen is a virus, viroid, bacterium, fungus, protozoan, algae, or helminth. In some embodiments, the pathogen is a fungus. In one embodiment, the pathogen is Coccidioides posadasii. In one embodiment, the disease or disorder is valley fever (coccidioidomycosis).

In one aspect, the present invention also provides methods for distinguishing a subject with an infectious disease or disorder from a subject without infectious disease or disorder. In one aspect, the present invention further provides methods relating to the proteins and antibodies of the invention that can be used to establish and evaluate treatment plans for a subject with an infectious disease or disorder.

In various embodiments, methods of diagnosing, or determining the prognosis of, a disease or disorder comprise detecting a pathogenic antigen, or an antibody that binds to a pathogenic antigen, in a sample obtained from a subject suspected of having a disease or disorder. In some embodiments, the pathogenic antigen is identified according to a method of the present disclosure. In some embodiments, the pathogenic antigen, or an antibody that binds to the pathogenic antigen, is detected by any conventional means known in the art including, but not limited to, immunological-based methods (e.g., enzyme-linked immunosorbent assays or lateral flow immunoassays), protein gel chromatography coupled with Western blotting, mass spectrometry (MS), protein gel chromatography coupled with MS, liquid chromatography (LC), LC coupled with UV detection, LC-MS, and LC-tandem MS (LC-MS/MS).

In some embodiments, the method comprises the steps of:
a) obtaining a sample from a subject suspected of having a disease or disorder;
b) contacting the sample with one or more proteins;
c) detecting any interactions of the proteins with one or more antibodies present in the sample; and
d) determining that the subject has the disease or disorder when an interaction is detected.

In some embodiments, the one or more proteins are immobilized. In some embodiments, the proteins are immobilized on a bead. In some embodiments, the proteins are immobilized on a planar solid substrate.

In some embodiments, the method comprises immobilizing an antibody and contacting proteins in the sample with the immobilized antibody.

In some embodiments, the method comprises the steps of:
a) obtaining a sample from a subject suspected of having a disease or disorder;
b) contacting the sample with one or more antibodies;
c) detecting any interactions of the antibodies with one or more proteins present in the sample; and
d) determining that the subject has the disease or disorder when an interaction is detected.

In some embodiments, the one or more antibodies are immobilized. In some embodiments, the antibodies are immobilized on a bead. In some embodiments, the antibodies are immobilized on a planar solid substrate.

In some embodiments, the interaction of the proteins with one or more antibodies is detected by optical, electrical, or physical means. In some embodiments, interaction of the proteins with one or more antibodies results in an optically observable (e.g., fluorescence, chemiluminescence) or electrical (e.g., voltage, current) change or alteration. In some embodiments, interaction of the antibodies with one or more antigens enables binding of a second antibody. In some embodiments, the secondary antibody is fluorescently labeled. In some embodiments, the second antibody possesses an enzymatic function, wherein the activity of the enzyme results in an optically observable phenomenon (e.g., change in color). In some embodiments, the second antibody is specific to a particular subset of antibodies. In some embodiments, the second antibody is an anti-IgG, anti-IgM, anti-IgA, or anti-IgE antibody.

In certain embodiments, the disclosure provides a method of diagnosing a subject with valley fever or determining the prognosis of the disease through the use of a diagnostic testing device of the present disclosure. In certain embodiments, the method comprises the steps of:
a) providing a diagnostic testing device according to the disclosure;
b) obtaining a sample (e.g., a serum sample) from the subject suspected of having or developing valley fever;
c) contacting the diagnostic testing device with the sample; and
d) detecting an interaction between the sample and the testing device.

Biological samples may be of any biological tissue or fluid. Frequently the sample will be a “clinical sample” which is a sample derived from a patient. The biological sample may contain any biological material suitable for detecting the desired proteins and antibodies, and may comprise cellular and/or non-cellular material obtained from the individual. A biological sample can be obtained by appropriate methods, such as, by way of examples, blood draw, fluid draw, biopsy, or surgical resection. Examples of biological samples include but are not limited to blood, lymph, urine, saliva, mucus, plasma, biological tissue, feces, gastrointestinal fluid, semen, and biopsies. Samples that are liquid in nature are referred to herein as “bodily fluids”. Body samples may be obtained from a patient by a variety of techniques including, for example, by scraping or swabbing an area or by using a needle to aspirate bodily fluids. Methods for collecting various body samples are well known in the art. Frequently, a sample will be a “clinical sample”, i.e., a sample derived from a patient. Such samples include, but are not limited to, bodily fluids which may or may not contain cells, e.g., blood (e.g., whole blood, serum or plasma), urine, saliva, tissue or fine needle biopsy samples, tissue sample obtained during surgical resection, and archival samples with known diagnosis, treatment, and/or outcome history. In some embodiments, the biological sample comprises a biological tissue of the subject, a blood sample of the subject, a bodily fluid sample of the subject, a fecal sample of the subject, a plasma sample of the subject, a saliva sample of the subject, a urine sample of the subject, or any combination thereof. In some embodiments, the biological sample comprises a serum sample.

In one embodiment, the method comprises analyzing the biological sample with an assay that specifically detects a biomarker. In some embodiments, the biomarker is a protein associated with Coccidioides posadasii infection, as described herein. In some embodiments, the biomarker is a protein of Table 1. In some embodiments, the biomarker is a protein selected from the group consisting of: woronin body major protein (A0A0E1S226), protein phosphatase 2C (J3K9P7), endochitinase-1/complement fixation (CF/CTS1/Endochitinase-1) (Q1E3R8), NADPH-cytochrome P450 reductase (J3KGJ4), proteasome component PUP2 (J3K500), ATP synthetase subunit 2, mitochondrial (J3KOX4), peroxisomal matrix protein (J3K6V5), elongation factor 1 gamma domain-containing protein (J3KLX5), 4-hydroxyphenylpyruvate dioxygenase (Q1E803), endo-1,3-beta-glucanase (J3KEN5), hsp90-like protein (J3KE37), ubiquitin-40S ribosomal protein S31 fusion protein (J3KC68), polyubiquitin (A0A0D8JXR9/A0A0D8JVA2), methylcrotonoyl-CoA carboxylase subunit beta (J3K8SO), calnexin (J3KHD0), GTP-binding protein YchF (A0A0E1RUI9), polyubiquitin (J3KHA0), 14-3-3-like protein (J3KA35), and hypothetical protein (titin/Nucleoporin homologue) (E9D7J3). In one embodiment, the method comprises analyzing the biological sample with an assay that specifically detects at least one biomarker. Examples of such assay include, but are not limited to: immunological-based methods (e.g., enzyme-linked immunosorbent assays or lateral flow immunoassays), protein gel chromatography coupled with Western blotting, mass spectrometry (MS), protein gel chromatography coupled with MS, liquid chromatography (LC), LC coupled with UV detection, LC-MS, and LC-tandem MS (LC-MS/MS). high-performance liquid chromatography (HPLC), ultra-performance liquid chromatography (UPLC), ultra-high-performance liquid chromatography (UHPLC), gas chromatography (GC), gas chromatography-mass spectrometry (GC-MS), globally optimized targeted mass spectrometry, targeted assay of about 200 metabolites, aqueous global profiling, liquid global profiling, GC-MS profiling, GC-MS flux analysis, carnitine analysis, lipid targeted analysis, quantitative lipid targeted analysis, tryptophan analysis, absolute quantification, multivariate statistical analysis, dynamic light scattering (DLS), nuclear magnetic resonance (NMR) spectroscopy, ultraviolet-visible (UV/Vis) spectroscopy, infrared (IR) spectroscopy, Raman spectroscopy, or any combination thereof.

In one embodiment, the method comprises using a multi-dimensional non-linear algorithm to determine if the level (e.g., activity, amount, concentration, concentration of the ionized form, concentration of the neutral form, expression, level, etc.) of a set of biomarkers in the biological sample is statistically different than a comparator. In some embodiments, the algorithm is drawn from the group consisting essentially of: linear or nonlinear regression algorithms; linear or nonlinear classification algorithms; ANOVA; neural network algorithms; genetic algorithms; support vector machines algorithms; hierarchical analysis or clustering algorithms; hierarchical algorithms using decision trees; kernel based machine algorithms such as kernel partial least squares algorithms, kernel matching pursuit algorithms, kernel fisher discriminate analysis algorithms, or kernel principal components analysis algorithms; Bayesian probability function algorithms; Markov Blanket algorithms; a plurality of algorithms arranged in a committee network; and forward floating search or backward floating search algorithms.

In one embodiment, the method comprises detecting one or more biomarkers in a biological sample of the subject. In some embodiments, the level of one or more of markers of the invention in the biological test sample of the subject is compared to a comparator. Non-limiting examples of comparators include, but are not limited to, a negative control, a positive control, standard control, standard value, an expected normal background value of the subject, a historical normal background value of the subject, a reference standard, a reference level, an expected normal background value of a population that the subject is a member of, or a historical normal background value of a population that the subject is a member of. In one embodiment, the comparator is a level (e.g., activity, amount, concentration, concentration of the ionized form, concentration of the neutral form, expression, level, etc.) of the one or more biomarker in a sample obtained from a subject not having an infectious disease or disorder. In one embodiment, the comparator is a level of the one or more biomarker in a sample obtained from a subject known not to have an infectious disease or disorder.

In one embodiment, the comparator is a level of the one or more biomarker in a sample obtained from a subject having a different infectious disease or disorder (e.g. fungal infection vs bacterial infection, fungal infection vs parasitic infection, fungal infection vs viral infection, etc.). In one embodiment, the comparator is a level of the one or more biomarker in a sample obtained from a subject known to have a different infectious disease or disorder (e.g. fungal infection vs bacterial infection, fungal infection vs parasitic infection, fungal infection vs viral infection, etc.).

In one aspect, the present invention includes methods for identifying subjects who have an infectious disease or disorder and subjects who do not have an infectious disease or disorder by detection of the biomarkers disclosed herein. In some embodiments, the biomarkers are used to generate a biomarker profile or signature of the subjects: (i) who have an infectious disease or disorder, and/or (ii) who do not have an infectious disease or disorder. In some embodiments, the biomarkers are used to generate a biomarker profile or signature of the subjects: (i) who have an infectious disease or disorder associated with fungal infection, (ii) who have an infectious disease or disorder associated with bacterial infection, (iii) who have an infectious disease or disorder associated with parasitic infection, (iv) who have an infectious disease or disorder associated with viral infection, (v) who do not have an infectious disease or disorder associated with fungal infection, (vi) who do not have an infectious disease or disorder associated with bacterial infection, (vii) who do not have an infectious disease or disorder associated with parasitic infection, (viii) who do not have an infectious disease or disorder associated with viral infection, or any combination thereof.

In one embodiment, the biomarker profile of a subject is compared to a predetermined or comparator biomarker profile or reference biomarker profile to identify an infectious disease or disorder. In one embodiment, the biomarker profile of a subject is compared to a predetermined or comparator biomarker profile or reference biomarker profile to diagnose an infectious disease or disorder. In one embodiment, the biomarker profile of a subject is compared to a predetermined or comparator biomarker profile or reference biomarker profile to assess the prognosis of an infectious disease or disorder. In one embodiment, the biomarker profile of a subject is compared to a predetermined or comparator biomarker profile or reference biomarker profile to evaluate the treatment of an infectious disease or disorder. In one embodiment, the biomarker profile of a subject is compared to a predetermined or comparator biomarker profile or reference biomarker profile to distinguish between different types of infectious diseases or disorders.

Control group samples may either be from a normal subject, samples from subjects with a known diagnosis of an infectious disease or disorder, or samples from subjects with no known diagnosis of an infection disease or disorder. As described below, comparison of the expression patterns of the sample to be tested with comparators can be used to identify or diagnose an infectious disease or disorder in the subject. In some instances, the control groups are only for the purposes of establishing initial cutoffs or thresholds for the assays of the invention. Therefore, in some instances, the systems and methods of the invention can identify or diagnose an infectious disease or disorder without the need to compare with a control group.

Information obtained from the methods of the invention described herein can be used alone, or in combination with other information (e.g., age, family history, disease status, disease history, vital signs, blood chemistry, expression of other gene signatures relevant to outcomes of an infectious disease or disorder, etc.) from the subject or from the biological sample obtained from the subject. In some embodiments, the biomarkers data is combined or correlated with other data or test results that include, but are not limited to measurements or results from serologic testing methods, enzyme immunoassay (EIA), complement fixation (CF), immunodiffusion, clinical presentation, serology, radiography, histology, culture, and clinical parameters or other algorithms for developing or having an infectious disease or disorder. In one embodiment, data include, but are not limited to age, ethnicity, other genomic data, and specific expression values of other gene signatures relevant to infection outcomes. In one embodiment, the data comprises subject information, such as medical history, travel history, and/or any relevant family history. Several serology techniques that can be used in combination with the compositions and methods of the present invention. Examples of serology techniques include, but are not limited to: ELISA, agglutination, precipitation, complement-fixation, fluorescent antibodies, and chemiluminescence.

In one aspect, the invention contemplates the detection of differentially expressed biomarkers using tissue microarray. In one embodiment, the method comprises diagnosing an infectious disease or disorder by detecting differentially expressed biomarkers in biological tissue excised from the subject during biopsy. In one aspect, the invention further contemplates using methods known to those skilled in the art to detect and to measure the level of one or more differentially expressed marker expression products.

In one embodiment, a cellular or fluid examination is used to detect or measure a variety of molecules including RNA, protein, and a number of molecules that are modified as a result of the protein's function. Exemplary diagnostic methods focusing on nucleic acids include but are not limited to amplification techniques, such as PCR and RT-PCR (including quantitative variants), and hybridization techniques, such as in situ hybridization, microarrays, and blots. Exemplary diagnostic methods focusing on proteins include but are not limited to binding techniques, such as ELISA, immunohistochemistry, microarray, and functional techniques, such as enzymatic assays.

The genes identified as being differentially expressed may be assessed in a variety of nucleic acid detection assays to detect or quantify the expression level of a gene or multiple genes in a given sample. For example, traditional Northern blotting, nuclease protection, RT-PCR, microarray, and differential display methods may be used for detecting gene expression levels. Methods for assaying for mRNA include Northern blots, slot blots, dot blots, and hybridization to an ordered array of oligonucleotides. Any method for specifically and quantitatively measuring a specific protein or mRNA or DNA product can be used. However, methods and assays are most efficiently designed with array or chip hybridization-based methods for detecting the expression of a large number of genes. Any hybridization assay format may be used, including solution-based and solid support-based assay formats.

The protein products of the genes identified herein can also be assayed to determine the amount of expression. Methods for assaying for a protein include Western blot, immunoprecipitation, and radioimmunoassay. The proteins analyzed may be localized intracellularly (most commonly an application of immunohistochemistry) or extracellularly (most commonly an application of immunoassays such as ELISA).

In some embodiments, the invention relates to different types of biomarkers (e.g., protein level or activity, nucleic acid level or activity, mRNA level or activity, gene expression, etc.) and their measurements that can be combined with the compositions and methods of the present invention. In various embodiments, the neutral form of the biomarkers is measured. In various embodiments, the derivative form of the biomarkers is measured. In various embodiments, the ionized form of the biomarkers is measured. In various embodiments, measurements of neutral biomarkers are used in conjunction with measurements of ionized biomarkers. Biomarkers generally can be measured and detected through a variety of assays, methods and detection systems known to one of skill in the art. Various methods include but are not limited to immunoassays, microarray, PCR, RT-PCR, refractive index spectroscopy (RI), ultra-violet spectroscopy (UV), fluorescence analysis, electrochemical analysis, radiochemical analysis, near-infrared spectroscopy (near-IR), infrared (IR) spectroscopy, nuclear magnetic resonance spectroscopy (NMR), light scattering analysis (LS), mass spectrometry, pyrolysis mass spectrometry, nephelometry, dispersive Raman spectroscopy, gas chromatography, liquid chromatography, gas chromatography combined with mass spectrometry, liquid chromatography combined with mass spectrometry, matrix-assisted laser desorption ionization-time of flight (MALDI-TOF) combined with mass spectrometry, ion spray spectroscopy combined with mass spectrometry, capillary electrophoresis, colorimetry and surface plasmon resonance. In this regard, biomarkers can be measured using the above-mentioned detection methods, or other methods known to the skilled artisan. Other biomarkers can be similarly detected using reagents that are specifically designed or tailored to detect them.

In some embodiments of the invention, methods of measuring biomarker levels in a biological sample obtained from a subject include, but are not limited to, an immunochromatography assay, an immunodot assay, a Luminex assay, an ELISA assay, an ELISPOT assay, a protein microarray assay, a ligand-receptor binding assay, displacement of a ligand from a receptor assay, displacement of a ligand from a shared receptor assay, an immunostaining assay, a Western blot assay, a mass spectrophotometry assay, a radioimmunoassay (RIA), a radioimmunodiffusion assay, a liquid chromatography-tandem mass spectrometry assay, an Ouchterlony immunodiffusion assay, reverse phase protein microarray, a rocket immunoelectrophoresis assay, an immunohistostaining assay, an immunoprecipitation assay, a complement fixation assay, FACS, an enzyme-substrate binding assay, an enzymatic assay, an enzymatic assay employing a detectable molecule, such as a chromophore, fluorophore, or radioactive substrate, a substrate binding assay employing such a substrate, a substrate displacement assay employing such a substrate, and a protein chip assay.

The concentration of the biomarker in a sample may be determined by any suitable assay. A suitable assay may include one or more of the following methods, an enzyme assay, an immunoassay, mass spectrometry, chromatography, electrophoresis or an antibody microarray, or any combination thereof. Thus, as would be understood by one skilled in the art, the systems and methods of the invention may include any method known in the art to detect a biomarker in a sample.

The invention described herein also relates to methods for a multiplex analysis platform. In one embodiment, the method comprises an analytical method for multiplexing analytical measurements of markers.

In various embodiments, the subject is a human subject, and may be of any race, ethnicity, sex, and age.

In certain embodiments, the method comprises: a) obtaining a serum sample from a subject suspected of having valley fever; b) contacting the serum sample with one or more proteins; c) detecting any interactions of the proteins with one or more antibodies present in the serum sample; and d) determining that the subject has valley fever when an interaction is detected. In some embodiments, the one or more proteins are selected from the group consisting of: woronin body major protein (A0A0E1S226), protein phosphatase 2C (J3K9P7), endochitinase-1/complement fixation (CF/CTS1/Endochitinase-1) (Q1E3R8), NADPH-cytochrome P450 reductase (J3KGJ4), proteasome component PUP2 (J3K500), ATP synthetase subunit 2, mitochondrial (J3KOX4), peroxisomal matrix protein (J3K6V5), elongation factor 1 gamma domain-containing protein (J3KLX5), 4-hydroxyphenylpyruvate dioxygenase (Q1E803), endo-1,3-beta-glucanase (J3KEN5), hsp90-like protein (J3KE37), ubiquitin-40S ribosomal protein S31 fusion protein (J3KC68), polyubiquitin (A0A0D8JXR9/A0A0D8JVA2), methylcrotonoyl-CoA carboxylase subunit beta (J3K8SO), calnexin (J3KHD0), GTP-binding protein YchF (A0A0E1RUI9), polyubiquitin (J3KHA0), 14-3-3-like protein (J3KA35), and hypothetical protein (titin/Nucleoporin homologue) (E9D7J3). In some embodiments, the one or more proteins are selected from the group consisting of: endochitinase-1/complement fixation (CF/CTS1/Endochitinase-1) (Q1E3R8), endo-1,3-beta-glucanase (J3KEN5), peroxisomal matrix protein (J3K6V5), hypothetical protein (titin/Nucleoporin homologue) (E9D7J3), hsp90-like protein (J3KE37), protein phosphatase 2C (J3K9P7), and polyubiquitin (A0A0D8JXR9/A0A0D8JVA2). In some embodiments, the one or more proteins are selected from the group consisting of: endochitinase-1/complement fixation (CF/CTS1/Endochitinase-1) (Q1E3R8), endo-1,3-beta-glucanase (J3KEN5), peroxisomal matrix protein (J3K6V5), and hypothetical protein (titin/Nucleoporin homologue) (E9D7J3).

In certain embodiments, the method comprises: a) obtaining a serum sample from a subject suspected of having valley fever; b) contacting the serum sample with one or more antibodies; c) detecting any interactions of the antibodies with one or more antigens present in the serum sample; and d) determining that the subject has valley fever when an interaction is detected. In some embodiments, the one or more antibodies target one or more proteins selected from the group consisting of: woronin body major protein (A0A0E1S226), protein phosphatase 2C (J3K9P7), endochitinase-1/complement fixation (CF/CTS1/Endochitinase-1) (Q1E3R8), NADPH-cytochrome P450 reductase (J3KGJ4), proteasome component PUP2 (J3K500), ATP synthetase subunit 2, mitochondrial (J3KOX4), peroxisomal matrix protein (J3K6V5), elongation factor 1 gamma domain-containing protein (J3KLX5), 4-hydroxyphenylpyruvate dioxygenase (Q1E803), endo-1,3-beta-glucanase (J3KEN5), hsp90-like protein (J3KE37), ubiquitin-40S ribosomal protein S31 fusion protein (J3KC68), polyubiquitin (A0A0D8JXR9/A0A0D8JVA2), methylcrotonoyl-CoA carboxylase subunit beta (J3K8S0), calnexin (J3KHD0), GTP-binding protein YchF (A0A0E1RUI9), polyubiquitin (J3KHA0), 14-3-3-like protein (J3KA35), and hypothetical protein (titin/Nucleoporin homologue) (E9D7J3). In some embodiments, the one or more antibodies target one or more proteins selected from the group consisting of: endochitinase-1/complement fixation (CF/CTS1/Endochitinase-1) (Q1E3R8), endo-1,3-beta-glucanase (J3KEN5), peroxisomal matrix protein (J3K6V5), hypothetical protein (titin/Nucleoporin homologue) (E9D7J3), hsp90-like protein (J3KE37), protein phosphatase 2C (J3K9P7), and polyubiquitin (A0A0D8JXR9/A0A0D8JVA2). In some embodiments, the one or more antibodies target one or more proteins selected from the group consisting of: endochitinase-1/complement fixation (CF/CTS1/Endochitinase-1) (Q1E3R8), endo-1,3-beta-glucanase (J3KEN5), peroxisomal matrix protein (J3K6V5), and hypothetical protein (titin/Nucleoporin homologue) (E9D7J3).

In some embodiments, the method further comprises step e) administering a treatment of the disease or disorder. In some embodiments, the disease or disorder is valley fever. In some embodiments, the treatment comprises administering an antifungal agent to the subject.

In some embodiments, step c) further comprises a step of washing the diagnostic testing device to remove unbound antibodies of the subject. In some embodiments, step d) comprises a step of contacting the diagnostic testing device with fluorescently-labeled secondary antibodies. In some embodiments, step d) further comprises a step of washing the device to remove unbound secondary antibodies. In some embodiments, step d) further comprises a step of reading the fluorescence output of each nanowell of the diagnostic testing device. In some embodiments, the method further comprises a step of determining that the subject has been infected with Coccidioides posadasii based on the fluorescence output. In some embodiments, the fluorescently-labeled secondary antibodies are one or more selected from the group consisting of anti-IgG, anti-IgM, anti-IgA, and anti-IgE antibodies.

In some embodiments, the subject is determined to have been infected with Coccidioides posadasii when the detection system produces an optical or electrical output. For example, the subject is determined to have been infected with Coccidioides posadasii when the interaction between one or more capture molecules and their corresponding antibodies in the sample obtained from the subject produce an optical or electrical output.

In some embodiments, the subject is determined to have been infected with Coccidioides posadasii when one or more capture proteins bind their corresponding antibody in the sample and produce an optical or electrical output. In some embodiments, the subject is determined to have been infected with Coccidioides posadasii when certain portion of the capture proteins bind their corresponding antibody. In some embodiments, the subject is determined to have been infected with Coccidioides posadasii when the ratio of capture molecules bound to their corresponding antibody is greater than capture proteins unbound. In some embodiments, the subject is determined to have been infected with Coccidioides posadasii when the ratio of capture molecules bound to their corresponding antibody is greater than a comparator. In some embodiments, the subject is determined to have been infected with Coccidioides posadasii when the one or more capture proteins and their corresponding antibody have increased antibody reactivity compared to the comparator. In some embodiments, the comparator is a subject or a mean value from a population of subjects known not to have been infected with valley fever.

In some embodiments, the subject is determined to have been infected with Coccidioides posadasii when antibody reactivity in the subject sample is increased at least 0.01 fold, at least 0.05 fold, at least 0.07 fold, at least 0.076 fold, at least 0.1 fold, at least 0.18 fold, at least 0.19 fold, at least 0.3 fold, at least 0.36 fold, at least 0.37 fold, at least 0.38 fold, at least 0.4 fold, at least 0.43 fold, at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 192 fold, at least 192.4 fold, at least 192.44 fold, at least 200 fold, at least 250 fold, at least 500 fold, or at least 1000 fold, or at least 10000 fold, when compared to a comparator.

In some embodiments, the subject is determined to have been infected with Coccidioides posadasii when at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or at least twenty of the antibodies against Coccidioides-associated proteins described herein, are detected in the sample of the subject.

In some embodiments, the subject is determined to have been infected with Coccidioides posadasii when at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or at least twenty of the Coccidioides-associated proteins described herein, are detected in the sample of the subject.

In some embodiments, the subject is determined to have been infected with Coccidioides posadasii when a certain portion of the nanowells produce a fluorescence output. In some embodiments, the subject is determined to have been infected when at least about 10%, at least about 20%, at least about 30%, at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or all of the nanowells produce a fluorescence output. In some embodiments, the nanowell is determined to be producing a fluorescence output when any fluorescence is detected. In some embodiments, the nanowell is determined to be producing a fluorescence output when the intensity of fluorescence is at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, at least about 220%, at least about 240%, at least about 260%, at least about 280%, at least about 300%, at least about 350%, at least about 400%, at least about 450%, at least about 500%, at least about 550%, at least about 600%, at least about 650%, at least about 700%, at least about 750%, at least about 800%, at least about 850%, at least about 900%, at least about 950%, at least about 1000%, at least about 1100%, at least about 1200%, at least about 1300%, at least about 1400%, at least about 1500%, at least about 1600%, at least about 1700%, at least about 1800%, at least about 1900%, at least about 2000%, at least about 3000%, at least about 4000%, or at least about 5000% greater than the corresponding fluorescence of an uninfected sample.

In some embodiments, the subject is an animal. In some embodiments, the subject is a mammal. In some embodiments, the subject is a dog. In some embodiments, the subject is a human.

In some embodiments, the methods are useful for determining the prognosis of a disease or disorder in a subject. In some embodiments, the results of the method indicate that a subject does not need intervening treatment. In some embodiments, results of the method indicate that the subject requires a specific treatment.

In some embodiments, the method further comprises administering a treatment of the disease or disorder to the subject. In some embodiments, the disclosure provides methods of treating a subject diagnosed as having valley fever. In some embodiments, the method comprises diagnosing the subject as having valley fever according to a diagnostic method of the disclosure and administering to the subject a therapeutically effective amount of an antifungal composition. In some embodiments, the antifungal composition comprises one or more selected from the group consisting of fluconazole, itraconazole, amphotericin B, voriconazole, posaconazole, isavuconazole, ketoconazole, terbinafine, nikkomycin Z, olorofim, and pharmaceutically acceptable salts and hydrates thereof. In some embodiments, the subject has previously been administered antibiotic treatment. In some embodiments, the subject does not respond to antibiotics.

In one aspect, the present invention relates to a method of distinguishing between a subject with an infectious disease or disorder and a subject without an infectious disease or disorder. In one aspect, the present invention relates to a method of distinguishing between different types of infectious diseases or disorders. In one embodiment, the method distinguishes between subjects with different types of infectious diseases or disorders. In one embodiment, the method distinguishes between a subject with a fungal infection and a subject with a bacterial infection. In one embodiment, the method distinguishes between a subject with a fungal infection and a subject with a parasitic infection. In one embodiment, the method distinguishes between a subject with a fungal infection and a subject with a viral infection. In one embodiment, the method distinguishes between a subject with a bacterial infection and a subject with a parasitic infection. In one embodiment, the method distinguishes between a subject with a bacterial infection and a subject with a viral infection. In one embodiment, the method distinguishes between a subject with a parasitic infection and a subject with a viral infection.

In some embodiments, the results of the diagnostic or prognostic method indicate that a specific antifungal composition should or should not be administered to the subject. In some embodiments, the results indicate a propensity of resistance to one or more antifungal compositions, thereby indicating the use of an alternative treatment.

In some embodiments, the results of the diagnostic or prognostic methods described herein indicate that a subject who has been treated with an anti-bacterial or an anti-viral treatment should be removed from the anti-bacterial or anti-viral treatment. In one embodiment, the subject should be removed from the anti-bacterial or anti-viral treatment and administered an ant-fungal agent or treatment, as described elsewhere herein.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the certain embodiments of the present invention, and are not to be construed as limiting in any way the remainder of the disclosure.

### Example 1: Immunoscreening for Coccidioides

The goal of this research is to define the sero-reactive landscape of all Coccidioides proteins. Based on the previously published proteome of the fungus (Coccidioides spp.) that causes Valley Fever (VF), a nucleic acid programmable protein array (NAPPA) is employed to transcribe and translate Coccidioidal proteins on the array so that they can be probed with anti-sera from individuals (dogs, humans, etc.) with VF.

### Array Preparation

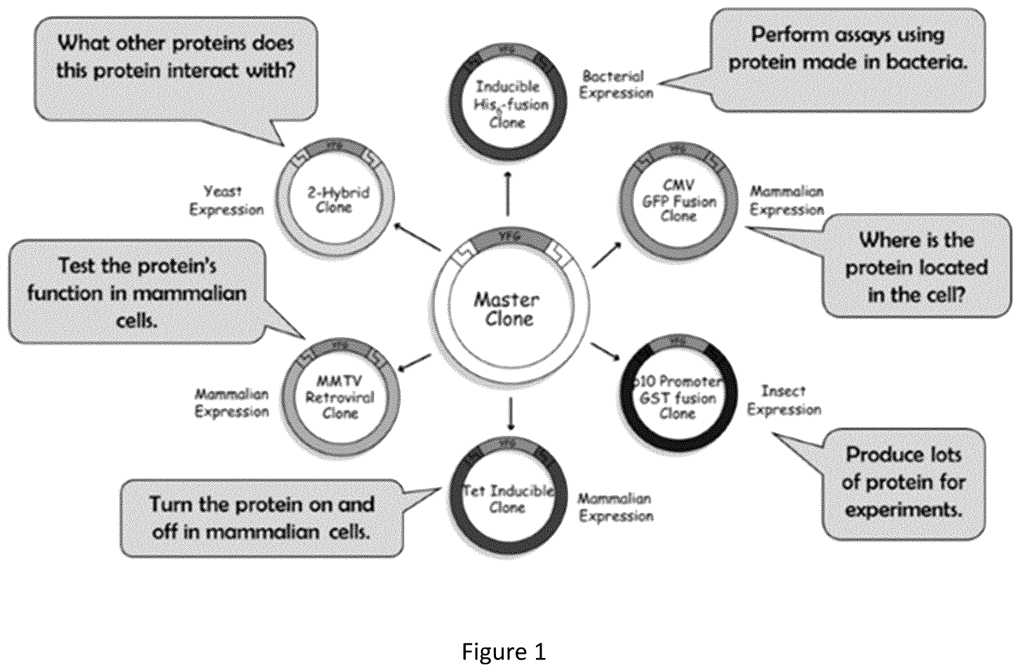

Spherule and mycelial extracts of Coccidioides posadasii were analyzed by liquid chromatography-tandem mass spectrometry (LC-MS/MS). The obtained peptide sequences were compared to known databases, revealing 603 coding regions. Additional constructs were selected based on review of the literature. Codon-optimized master DONR/expression plasmids were prepared for facile generation of plasmids containing each of the genes. The master plasmids allow for rapid generation of a variety plasmids suitable for different conditions, including yeast-2-hybrid assays for protein interactions, green fluorescent protein (GFP)-fusion for proteins for tracking protein localization, His-tag-fusion for rapid protein production and purification, inducible promotors for temporal examination, and glutathione S-transferase (GST)-fusion proteins for protein purification and immobilization (FIG. 1).

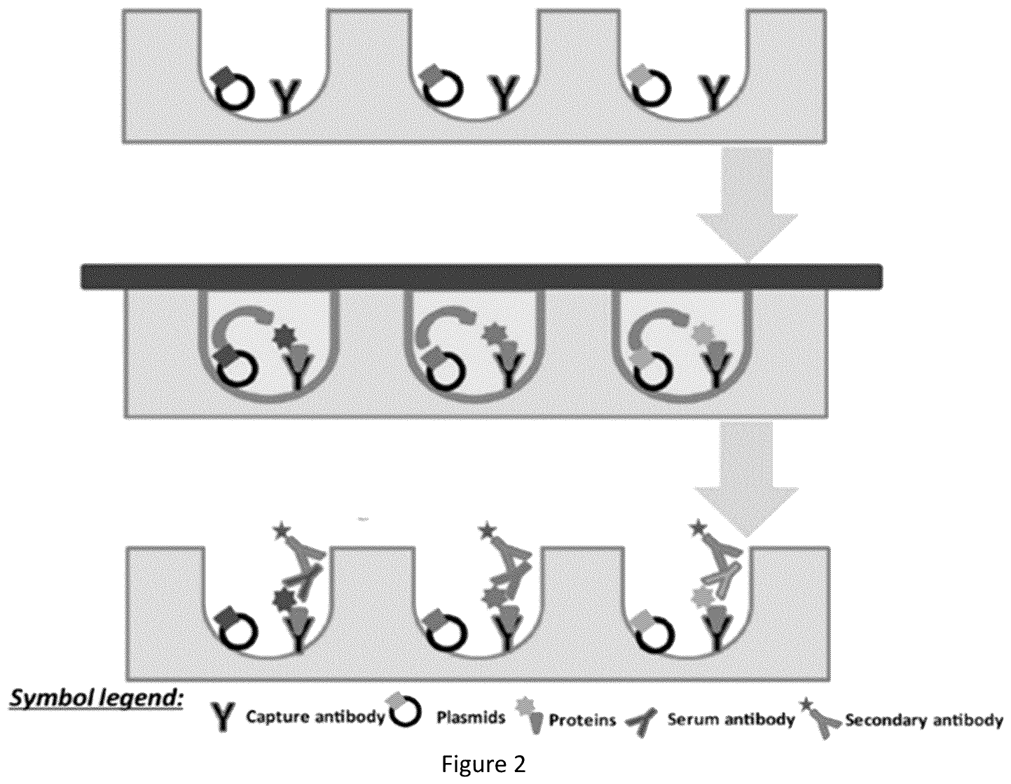

For the present assay, the genes for each of the proteins were cloned into a GST-fusion plasmid. The plasmid constructs are printed into nanowells of the high-density NAPPA platform along with antibodies to the GST tag (FIG. 2, top). Briefly, plasmids are admixed in a printing matrix of BSA, heterobifunctional cross-linker, and anti-GST antibodies. The resulting assays are stable under suitable storage conditions for extended periods.

### Serum Sample Set

For identifying which proteins are antigenic, several different types of samples were examined. A set of Coccidioides-infected samples was obtained from a variety of breeds which had community-acquired infections (n=10). A control (noninfected) set of samples was obtained from a variety of breeds identified as not be infected with Coccidioides by complement fixation (CF, n=7). A second set of Coccidioides-infected samples was obtained from dogs with laboratory-acquired disease. In this dataset a single breed of dogs (beagles, n=12) was used, which were infected with a single strain of Coccidioides at controlled dosages. These animals also provided samples at a variety of timepoints (0, 2-, 4-, 6-, and 8-weeks post-infection).

### Immunogenic Assay of Samples

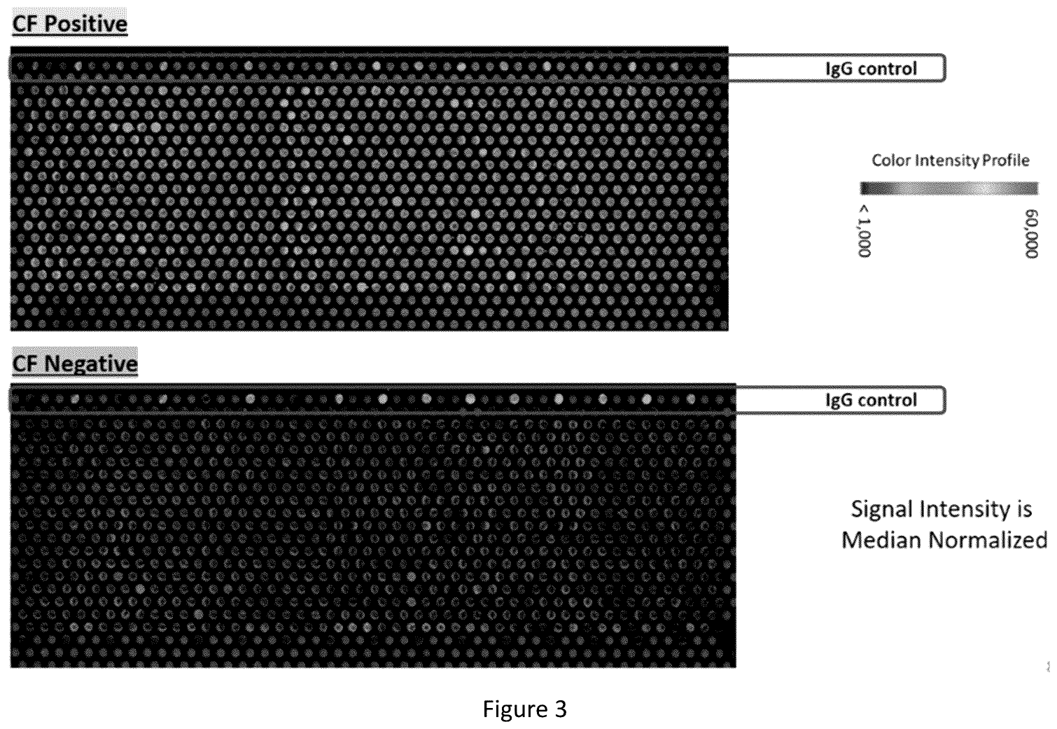

On the day of testing, coupled in vitro transcription/translation assays are performed to produce the proteins in the nanowells using a HeLa cell lysate (FIG. 2, middle). The proteins are individually captured in their targeted nanowell by the anti-GST antibody and are displayed for reactivity to immune or control sera. Binding of antibodies is detected by incubation with a fluorescently-labeled secondary antibody (FIG. 2, bottom). After washing, to remove non-reacted fluorescent antibodies, the slides for CF-positive and CF-negative samples are scanned by a microarray scanner to digitize the fluorescent intensity level for subsequent statistical analyses (FIG. 3). Proteins on the array are identified as antigens by reacting serum from infected individuals (separately) followed by detection of antibodies bound to the proteins using anti-serum labeled with a fluorescent molecule (anti-canine IgG-fluor). After each individual serum sample is run on the array, 51 commonly sero-reactive proteins are identified, 20 of which are preliminary candidates (Table 1).

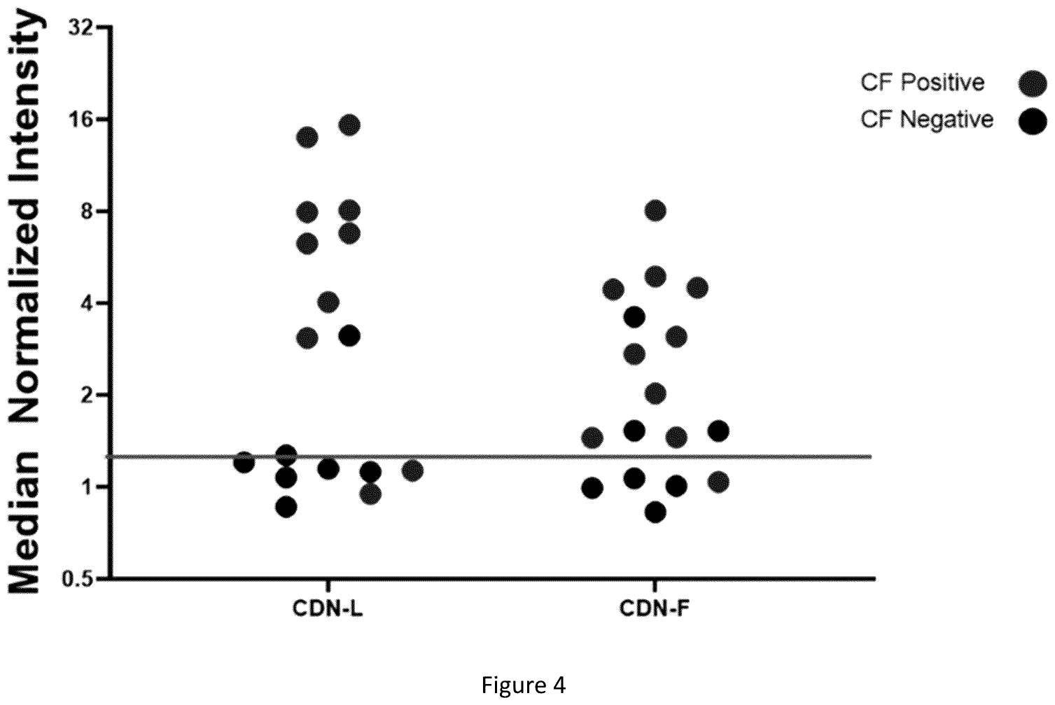

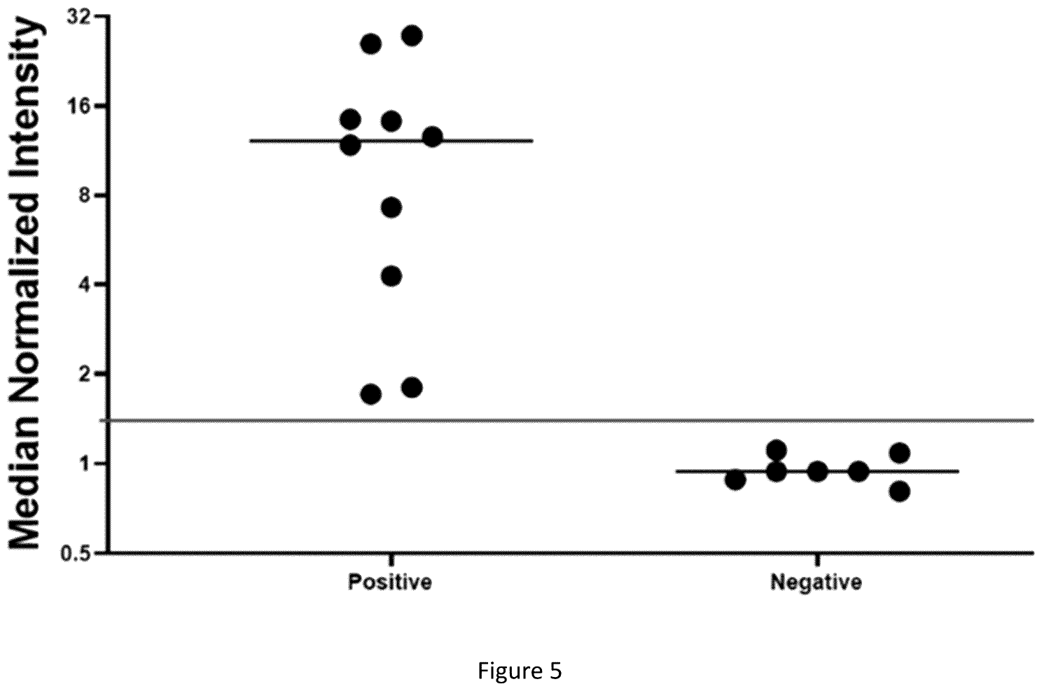

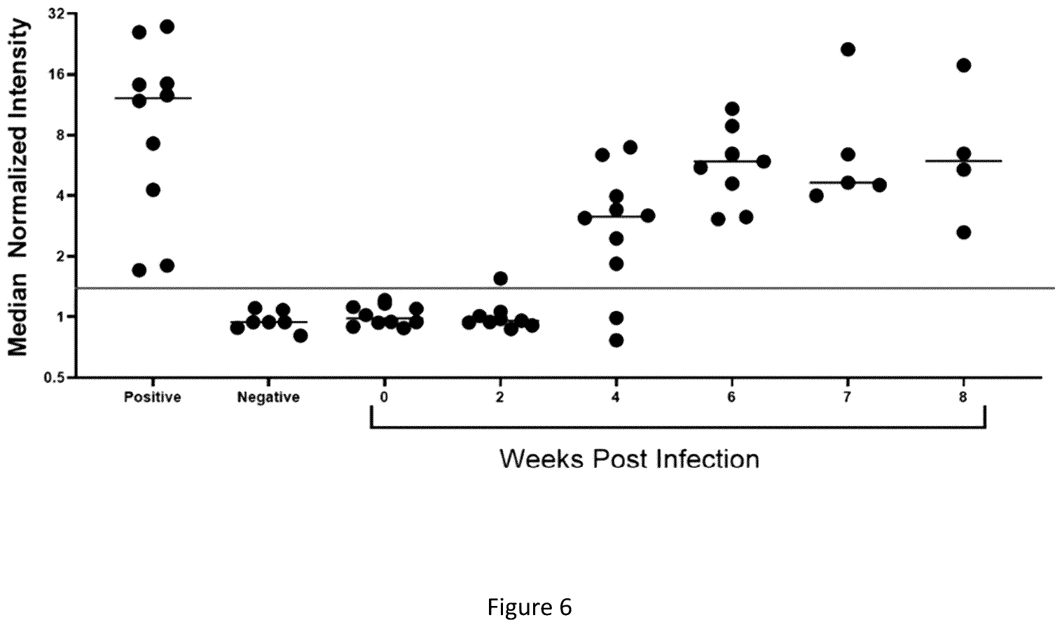

Examination of the antigen reactivities of the community-acquired infection samples compared and control samples demonstrated no difference in reactivity to coccidioidin fractionation (CDN-F). There was, however, a difference between the reactivity of CF-positive and CF-negative serum with coccidioidin lysate (CDN-L), with CF-positive samples grouping together with higher immunoreactivity compared to CF-negative samples (FIG. 4). As proof of concept, direct comparison of immunoreactivity to the NAPPA complement fixation antigen (CTS-1) demonstrated the average fluorescence intensity in CF-positive samples was more than 12-times that of CF-negative samples (FIG. 5). Examination of the time course data provided by the laboratory-acquired infection samples demonstrated that immunoreactivity to CTS-1 did not occur in serum samples taken less than 4-weeks post-infection (FIG. 6).

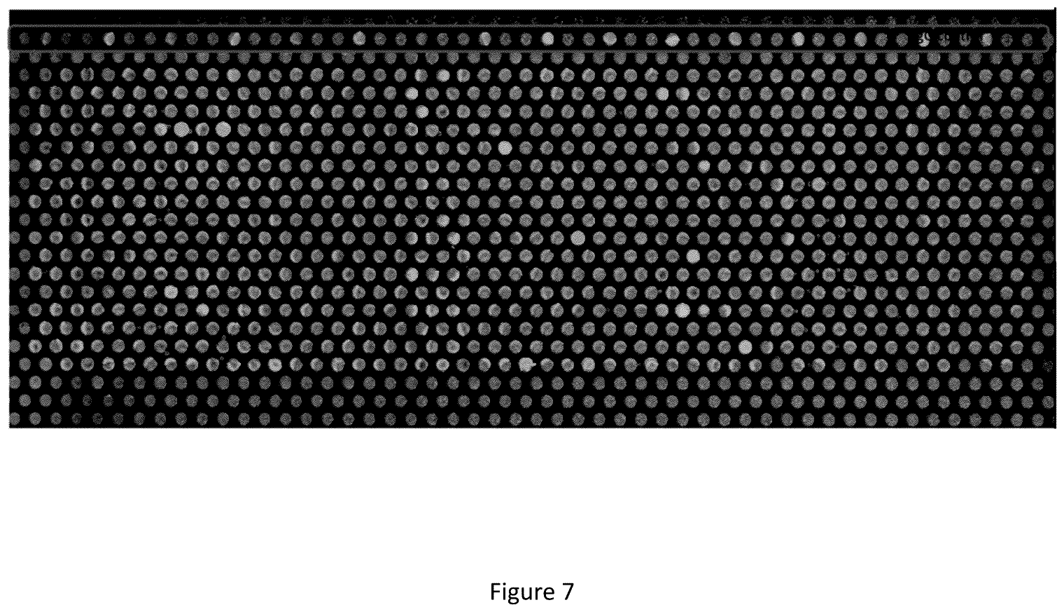

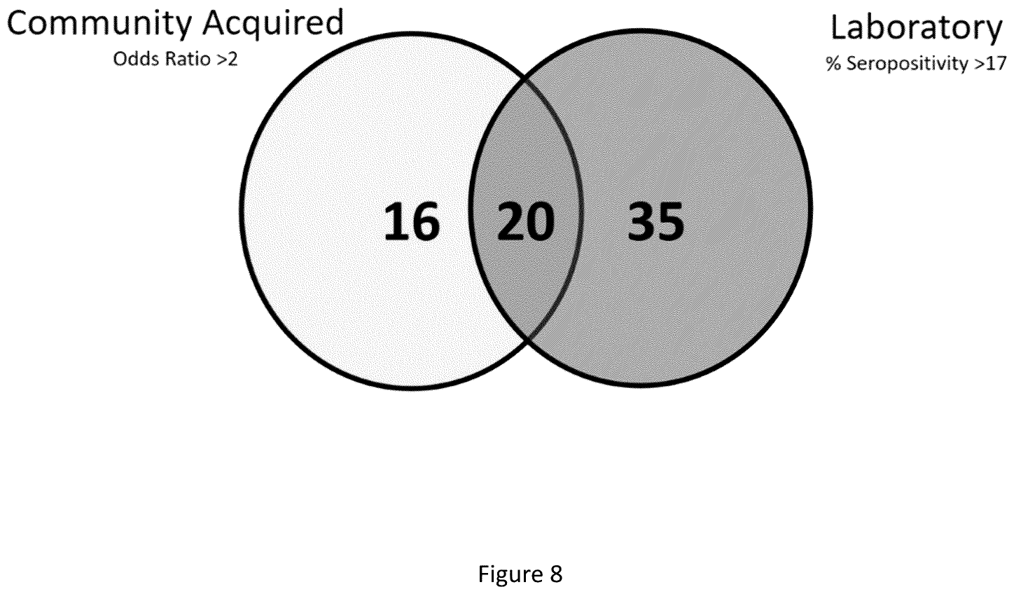

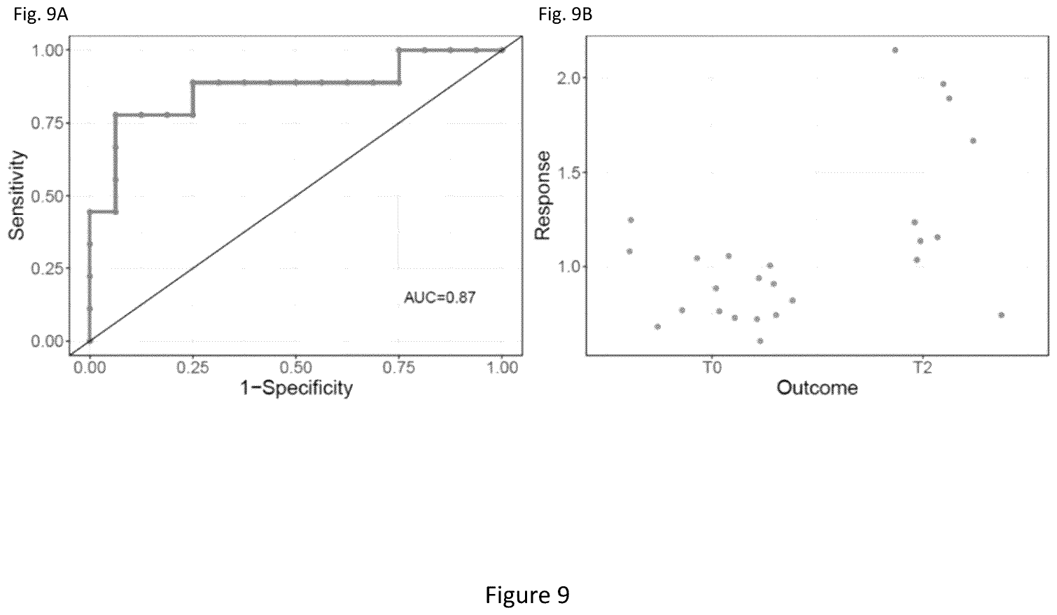

Examining the antigen hits from the community-acquired and laboratory-acquired samples, a total of 71 antigens were observed, with 20 being commonly observed between the two samples (FIG. 7 and FIG. 8). Using a diagnostic model with one of these antigens (hypothetical protein CIMG_07502, Accession No. E9D7J3) it was possible to identify infection earlier than with CTS-1. Early responders to infection demonstrated significant response to the antigen as early as 2-weeks post-infection (FIG. 9).

### Example 2: Human and Animal Coccidioides Reactive Proteins

Most current diagnostic tests for Coccidioidomycoisis (also known as Valley Fever, VF) utilize complex fungal extracts to perform immunological assays. The fungal extracts are notoriously variable in quality and quantity of individual components due to the heterogenous nature of production. Moreover, the current diagnostic tests have low sensitivity and specificity for fungal antigens. Sample analysis is further complicated by the fact that antibody levels are low at the initial early presentation of infection.

TABLE 2
Primary Antigen Candidates

| | | | |
|---|---|---|---|
| CiCD00966787 | CIMG_02795 | CF/CTS1/ | XP_001249024.1 |
| | | Endochitinase-1 | |
| CiCD00967147 | CIMG_04729 | hsp90-like protein | XP_001245288.1 |
| CiCD00967130 | CIMG_04988 | endo-1,3-beta- | XP_001245547.2 |
| | | glucanase | |
| CiCD00966958 | CIMG_05828 | peroxisomal | XP_001241932.1 |
| | | matrix protein | |
| CiCD00966779 | CIMG_07139 | protein phosphatase | XP_001243243.2 |
| | | 2C | |
| CiCD00967199 | CIMG_11455 | polyubiquitin | XP_004446139.1 |
| CpCD00966874 | CPSG_05795 | hypothetical protein | EFW17352.1 |

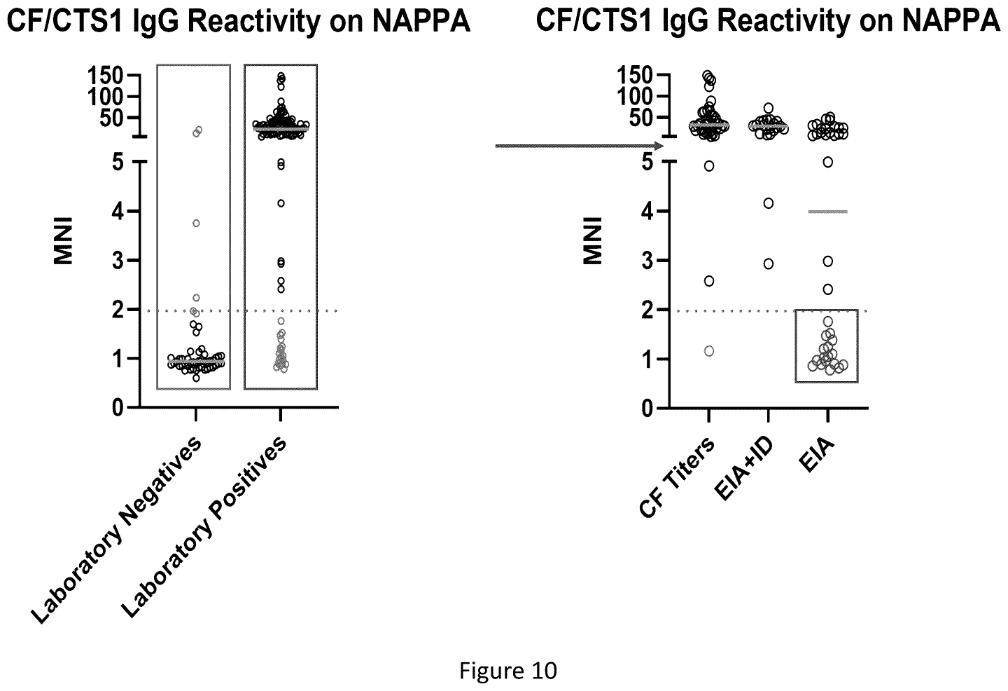

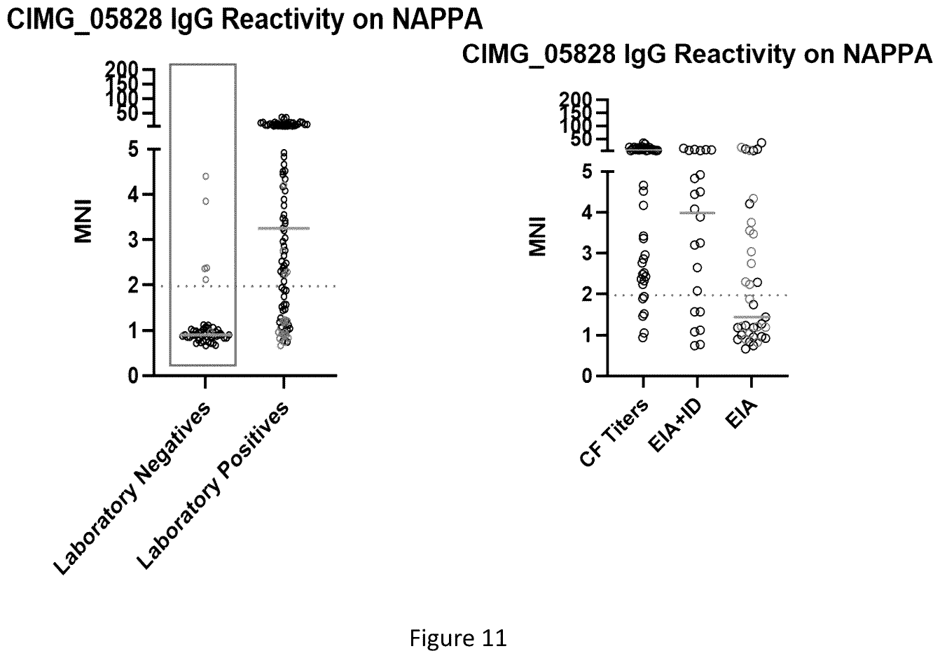

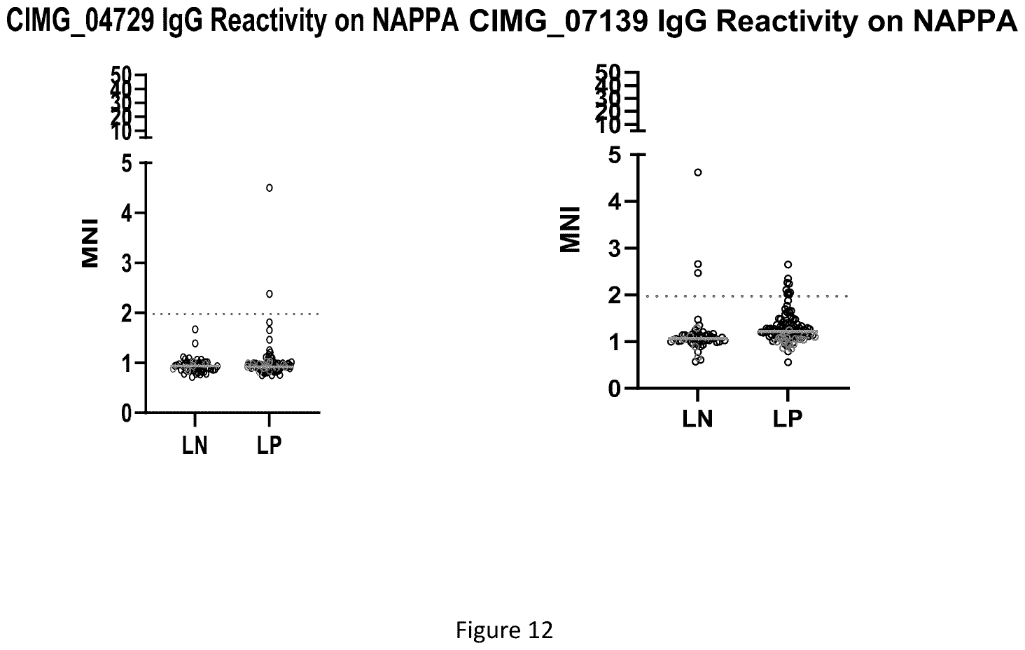

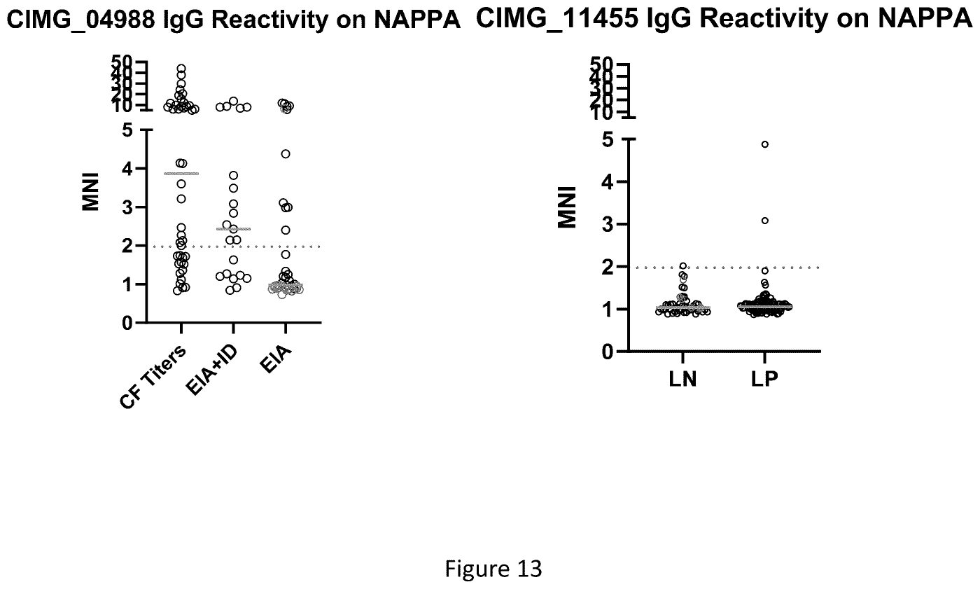

The antibody reactivity of the 7 candidate antigens in Table 2 were analyzed by the traditional complement fixation test, ELISA (Enzyme ImmunoAssay, EIA), or Immunodiffusion, (ID)). CF/CTS1 (FIG. 10), J3K6V5 (FIG. 11), J3KE37 (FIG. 12, left), J3K9P7 (FIG. 12, right), J3KEN5 (FIG. 13, left), and A0A0D8JXR9/A0A0D8JVA2 (FIG. 13, right) IgG reactivity on NAPPA was measured.

These proteins, among others, are used to construct a multi-antigen test to detect antibodies against Coccidioides species, which aids in the diagnosis of VF and prognostic monitoring of patients (dog, human, etc.) with Valley Fever. This technique further allows rapid identification of proteins that react with IgG, IgM, or even IgA or IgE, in any species infected with Coccidioides. In screening of canine sera 51 Coccidioides proteins have been identified that react with IgG from infected dogs. These proteins can be used alone or combined with previously known sero-reactive proteins to develop a multi-antigen diagnostic and prognostic test for VF.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of testing a subject for valley fever comprising the steps of:
a) obtaining a serum sample from the subject;
b) contacting the serum sample with one or more proteins selected from the group consisting of: woronin body major protein (A0A0E1S226), protein phosphatase 2C (J3K9P7), endochitinase-1/complement fixation (CF/CTS1/Endochitinase-1) (Q1E3R8), NADPH-cytochrome P450 reductase (J3KGJ4), proteasome component PUP2 (J3K500), ATP synthetase subunit 2, mitochondrial (J3KOX4), peroxisomal matrix protein (J3K6V5), elongation factor 1 gamma domain-containing protein (J3KLX5), 4-hydroxyphenylpyruvate dioxygenase (Q1E803), endo-1,3-beta-glucanase (J3KEN5), hsp90-like protein (J3KE37), ubiquitin-40S ribosomal protein S31 fusion protein (J3KC68), polyubiquitin (A0A0D8JXR9/A0A0D8JVA2), methylcrotonoyl-CoA carboxylase subunit beta (J3K8SO), calnexin (J3KHD0), GTP-binding protein YchF (A0A0E1RUI9), polyubiquitin (J3KHA0), 14-3-3-like protein (J3KA35), and hypothetical protein (titin/Nucleoporin homologue) (E9D7J3);
c) detecting an interaction between an antibody in the serum and the one or more proteins; thereby detecting the presence of the antibody in the serum of the subject; and
d) determining that the subject has valley fever when an interaction is detected.

2. The method of claim 1, wherein the one or more proteins are selected from the group consisting of: endochitinase-1/complement fixation (CF/CTS1/Endochitinase-1) (Q1E3R8), endo-1,3-beta-glucanase (J3KEN5), peroxisomal matrix protein (J3K6V5), hypothetical protein (titin/Nucleoporin homologue) (E9D7J3), hsp90-like protein (J3KE37), protein phosphatase 2C (J3K9P7), and polyubiquitin (A0A0D8JXR9/A0A0D8JVA2).

3. The method of claim 1, wherein the one or more proteins are selected from a group consisting of: endochitinase-1/complement fixation (CF/CTS1/Endochitinase-1) (Q1E3R8), endo-1,3-beta-glucanase (J3KEN5), peroxisomal matrix protein (J3K6V5), and hypothetical protein (titin/Nucleoporin homologue) (E9D7J3).

4. The method of claim 1, wherein in step b) the serum sample is contacted with a plurality of the one or more proteins.

5. The method of claim 1, wherein it is determined that the subject has valley fever when at least one of the one or more proteins interact with an antibody in the serum sample.

6. The method of claim 1, wherein it is determined that the subject has valley fever when an increase in the amount of the antibody is detected, relative to a reference level.

7. The method of claim 1, wherein the interaction between the serum and the one or more proteins is detected using a lateral flow immunoassay, an Enzyme-Linked Immunosorbent Assay (ELISA), an immunodiffusion assay, an immunosensor assay, a Nucleic Acid Programmable Protein Array (NAPPA), and an immunofluorescence assay.

8. The method of claim 1, wherein the method further comprises step e) administering a therapeutically effective amount of an antifungal composition to the subject.

9. The method of claim 8, wherein the antifungal composition comprises one or more selected from the group consisting of fluconazole, itraconazole, amphotericin B, voriconazole, posaconazole, isavuconazole, ketoconazole, terbinafine, nikkomycin Z, olorofim, and pharmaceutically acceptable salts and hydrates thereof.

10. A diagnostic testing device for detecting Valley fever comprising a solid substrate decorated with a plurality of proteins, wherein the proteins are one or more proteins associated with Coccidioides posadasii infection.

11. The diagnostic testing device of claim 10, wherein the one or more proteins are selected from the group consisting of: woronin body major protein (A0A0E1S226), protein phosphatase 2C (J3K9P7), endochitinase-1/complement fixation (CF/CTS1/Endochitinase-1) (Q1E3R8), NADPH-cytochrome P450 reductase (J3KGJ4), proteasome component PUP2 (J3K500), ATP synthetase subunit 2, mitochondrial (J3KOX4), peroxisomal matrix protein (J3K6V5), elongation factor 1 gamma domain-containing protein (J3KLX5), 4-hydroxyphenylpyruvate dioxygenase (Q1E803), endo-1,3-beta-glucanase (J3KEN5), hsp90-like protein (J3KE37), ubiquitin-40S ribosomal protein S31 fusion protein (J3KC68), polyubiquitin (A0A0D8JXR9/A0A0D8JVA2), methylcrotonoyl-CoA carboxylase subunit beta (J3K8SO), calnexin (J3KHD0), GTP-binding protein YchF (A0A0E1RUI9), polyubiquitin (J3KHA0), 14-3-3-like protein (J3KA35), and hypothetical protein (titin/Nucleoporin homologue) (E9D7J3).

12. The diagnostic testing device of claim 11, wherein the one or more proteins are selected from the group consisting of: endochitinase-1/complement fixation (CF/CTS1/Endochitinase-1) (Q1E3R8), endo-1,3-beta-glucanase (J3KEN5), peroxisomal matrix protein (J3K6V5), hypothetical protein (titin/Nucleoporin homologue) (E9D7J3), hsp90-like protein (J3KE37), protein phosphatase 2C (J3K9P7), and polyubiquitin (A0A0D8JXR9/A0A0D8JVA2).

13. The diagnostic testing device of claim 11, wherein the one or more proteins are selected from a group consisting of: endochitinase-1/complement fixation (CF/CTS1/Endochitinase-1) (Q1E3R8), endo-1,3-beta-glucanase (J3KEN5), peroxisomal matrix protein (J3K6V5), and hypothetical protein (titin/Nucleoporin homologue) (E9D7J3).

14. The diagnostic testing device of claim 12, wherein the solid substrate is decorated with a plurality different proteins.

15. The diagnostic testing device of claim 12, wherein the device is used in an assay selected from the group consisting of: a lateral flow immunoassay, an Enzyme-Linked Immunosorbent Assay (ELISA), an immunodiffusion assay, an immunosensor assay, a Nucleic Acid Programmable Protein Array (NAPPA), and an immunofluorescence assay.

16. A method of identifying antigens associated with a pathogen comprising the steps of:
a) obtaining a number of serum samples from subjects infected with the pathogen of interest and a number of serum samples from uninfected control subjects;
b) contacting the serum samples with a plurality of proteins expressed by the pathogen of interest;
c) detecting interactions between the proteins expressed by the pathogen of interest and antibodies present in the serum samples; and
d) determining that a protein expressed by the pathogen of interest is an antigen when the protein interacts with antibodies present in the infected samples but not the uninfected samples.

17. The method of claim 16, wherein the plurality of proteins are immobilized on a solid substrate.

18. The method of claim 16, wherein in step c) interactions between the proteins and antibodies are detected by contacting the proteins and antibodies with fluorescently labeled secondary antibodies.

19. The method of claim 17, wherein the solid substrate comprises a plurality of different proteins expressed by the pathogen of interest.
